(19) United States
(12) Patent Application Publication
(10) Pub. No.: US 2025/0387565 A1
(43) Pub. Date: Dec. 25, 2025

(54) AUTO-INFLATABLE PATCH ASSEMBLY FOR DELIVERY OF THERAPEUTIC AGENTS TO AN INTESTINAL WALL

(71) Applicant: ALMA THERAPEUTICS LTD, Kfar-Saba (IL)

(72) Inventors: Anat SOFER RAZ, Herot (IL); Oz CABIRI, Hod Hasharon (IL); Noa GRANADA, RISHON LEZION (IL); Yossi GROSS, Mazor (IL)

(21) Appl. No.: 19/105,361

(22) PCT Filed: Aug. 22, 2023
(86) PCT No.: PCT/IB2023/058358

§ 371 Date: Feb. 21, 2025

Related U.S. Application Data
(60) Provisional application No. 63/576,454, filed on Aug. 22, 2022.

(51) Int. Cl. A61M 5/155; A61M 5/142; A61M 5/145; A61M 5/158; A61M 31/00

(52) U.S. Cl. CPC A61M 5/155; A61M 5/14276; A61M 5/158; A61M 31/002; A61M 2005/14284; A61M 2005/14513; A61M 2207/00; A61M 2210/106

(57) ABSTRACT

Embodiments of the invention provide an auto-inflatable patch assembly for drug delivery within a lumen, having: a pliable and inflatable patch having water and gas-impermeable walls, which define an internal chamber and an array of penetrating needles disposed on a surface thereof; one or more auxiliary gas-generating reaction chambers distinguished from and fluidly connected to the inflatable patch, said reaction chamber defined by pliable reaction chamber walls that house a water-sensitive gas-generating formulation; said reaction chamber walls having at least a portion being a water-permeable outer surface, wherein the patch assembly is configured such that each gas-generating formulation or a set thereof, is retained within its respective auxiliary gas-generating reaction chamber during inflation.

### RELATED APPLICATIONS

The present patent application claims the benefit of U.S. application Ser. No. 17/892,235, which was filed on Aug. 22, 2022, and which is incorporated herein by reference in its entirety.

### FIELD OF THE INVENTION

Embodiments of the present disclosure relate to medical devices for auto-inflation driven by exposure to water.

### BACKGROUND OF THE INVENTION

Orally administered, intestinal wall drug delivery is an area of interest because of the potential of delivering therapeutic agents with a high relative bioavailability due to increased blood flow, the possibility of locally delivering therapeutic agents to a site in the intestine, and the convenience of the oral application route for improved patient compliance, particularly in the case of biologics (proteins, peptides, etc.) which are typically delivered by parenteral routes. An orally administered transintestinal patch that does not involve pain can deliver a therapeutic agent to the blood while also enabling smooth, consistent, and safe penetration of a therapeutic agent through the intestinal wall. This is especially interesting for some therapeutic agents, including antibodies, proteins, and peptides, which are currently not delivered orally due to the sensitivity of the molecules to gastric pH, enzymatic degradation in the GI tract, presence of mucus, as well as the low permeation characteristics of the intestine wall for the size and polarity of this group of molecules.

Although progress has been made in this field, several challenges still hinder development. Many technologies rely on an enteric-coated capsule that contains a delivery device, incorporating a deployment mechanism with penetrating needles and a therapeutic agent. One major challenge is the effectiveness of the deployment of penetrating needles, which is reliant on the control and influence of intestinal fluid.

A common approach involves the release of the capsule in the intestinal fluid, which subsequently initiates a chemical reaction between a first and second reactant (such as citric acid and bicarbonate) to produce gas (like carbon dioxide). Typically, these reactants remain separate throughout the product's shelf life and initial stages post-administration, only reacting once the device is exposed to the intestinal environment. In this way, the gas production is delayed until after swallowing and deployment of the device in the intestine. The gas production then drives the actuation of the penetrating needle into the intestinal lumen wall where it can deliver a therapeutic.

Several different methods exist for separating a first and second reactant, for example, employing a water-sensitive disintegrating separation means. U.S. Pat. No. 9,149,617 discloses a mechanism by which a liquid and two reactants are kept in separate compartments. Exposure to intestinal fluid causes a separation means or valve to open. Two reactants, typically citric acid, and bicarbonate can then mix with the water and produce a gas such as carbon dioxide in an effervescent reaction. This gas then expands the inflatable patch, pushing a penetrating needle into the intestine wall. U.S. Pat. No. 9,492,396 discloses sodium bicarbonate, which at least partially coats a surface of a medication-delivery element, that releases a gas that promotes unrolling of the elongated medication-delivery element after exposure to intestinal fluid.

Additional challenges relate to the contradicting material requirements for a surface configured to constrain gas to accommodate balloon-like inflation while promoting water absorption to deliver fluid to the effervescent reactants. Using distinguished materials results in a seam that have been found to leak gas.

Developing a pharmaceutically acceptable device with a gas-generating mechanism for inflating an inflatable patch is a complex task due to the conflicting goals involved. The intestinal environment must trigger the device to ensure the gas generation occurs at the desired, time, location and position within the GI tract. At the same time, the device must be stable enough to withstand humid environments for shelf-life purposes, be powered for gas inflation, and maintain its durability during deployment in a lumen, especially in the intestine. While research has progressed, there is still a need for improved gas-generating devices and methods to drive inflation and delivery of therapeutic agents through the intestinal lumen wall.

### SUMMARY OF THE INVENTION

In a first aspect, there is provided an auto-inflatable patch assembly (herein “patch assembly”) for drug delivery within a lumen (e.g., GI tract lumen), comprising: a pliable and inflatable patch having water and gas-impermeable walls (“inflatable patch walls”), which define an internal chamber and an array of penetrating needles disposed on a surface thereof. In various embodiments, one or more auxiliary gas-generating reaction chambers arc distinguished from and fluidly connected to the inflatable patch, said reaction chamber defined by pliable reaction chamber walls that house a water-sensitive gas-generating formulation; said reaction chamber walls being configured to constrain gas (i.e., substantially gas impermeable) and having at least a portion being a water-permeable outer surface, wherein the patch assembly may be configured such that each gas-generating formulation (or a set of formulations), is retained within its respective auxiliary gas-generating reaction chamber during inflation.

In another aspect, there is provided an auto-inflatable patch assembly for drug delivery within a lumen, comprising: a pliable and inflatable patch having water and gas-impermeable walls, which define an internal chamber and an array of penetrating needles disposed on a surface thereof; one or more auxiliary gas-generating reaction chambers distinguished from and fluidly connected to the inflatable patch, said reaction chamber defined by pliable reaction chamber walls that house water-sensitive gas-generating formulations; said reaction chamber walls configured to constrain gas and having at least a portion being a water-permeable outer surface, wherein the patch assembly is configured to apply a force per area of between 0.2 and 1.0 or between 0.3 and 0.7 N/cm².

In various embodiments, the patch assembly may be compressed and further housed within a swallowable enteric outer shell. In this case, the patch assembly is configured for delivery of a therapeutic agent into an intestinal wall (e.g., small intestinal wall).

In various embodiments, the water-sensitive gas-generating formulation may be an extended-release gas-generating formulation. For example, the extended-release gas-generating formulation may include a viscosity enhancer.

In various embodiments, the gas-generating reaction chamber may be configured to produce a consistent amount of gas over an extended period, for example more than 30 minutes. In various embodiments, the water-sensitive gas-generating formulation may be configured to generate gas to provide a pressure of the inflatable patch as measured at 37° C. of more than 3 psi. In various embodiments, the water-sensitive gas-generating formulation may be configure d to generate gas to provide a substantially consistent pressure of the inflatable patch as measured at 37° C. for about 30 minutes. In various embodiments, the water-sensitive gas-generating formulation may be configured to generate gas to provide a substantially consistent pressure of the inflatable patch of more than 2 psi as measured at 37° C. for about 30 minutes.

In various embodiments, one or more auxiliary gas-generating reaction chambers are fluidly connected to the internal chamber but separated from the inflatable patch by a formulation-retentive element. In various embodiments, one or more auxiliary gas-generating reaction chambers are fluidly connected to the internal chamber but distinguished from the inflatable patch by a water-permeable surface and housing of the water-sensitive gas-generating formulation therein (which is not present in the inflatable patch).

In various embodiments, the patch assembly may be configured so that a combination of the internal chamber of the patch and respective interiors of one or more auxiliary gas reaction chambers are configured to constrain gas within.

In various embodiments, the first and second reactants are housed within a single compartment.

In various embodiments, the gas-generating reaction chamber may be positioned along an outer circumference, perimeter, or side surface of the inflatable patch. For example, there may be two or more gas-generating reaction chambers positioned along an outer circumference, perimeter, or side surface of the inflatable patch. In another example, there may be three or more gas-generating reaction chambers positioned along an outer circumference, perimeter, or side surface of the inflatable patch.

In various embodiments, a formulation-retentive element may be positioned along an inflatable patch outer circumference, perimeter, outer or side surface. The formulation-retentive element may be designed to permit fluid passage while restricting the passage of the water-sensitive gas-generating formulation, typically based on a geometric selection although other methods may be envisioned.

In various embodiments, at least 50% of the outer surface of the reaction chamber wall is water-permeable or preferably directionally permeable i.e., from the outside towards the internal volume of the reaction chamber.

In various embodiments, the penetrating needles have a length of more than 6.0, mm or alternatively, between 1.4 and 2.8 mm. In some embodiments, the penetrating needles further comprise a therapeutic agent. In some embodiments, the inflatable patch comprises a fluid therapeutic agent operably connected to a penetrating needle base.

The auto-inflatable patch assembly of any one of claims 1 to 27, wherein the array of penetrating needles are positioned on a first surface of the inflatable patch. For example, the first surface may be a planar surface of the inflatable patch in its uninflated expanded state. In some embodiments, the array of penetrating needles are positioned on the first surface of the inflatable patch, and the inflatable patch further comprises an opposing wall having a surface, wherein the surface has no penetrating needles.

In various embodiments, the inflatable patch has a maximum dimension of less than 9 cm such as 5.5 cm. In some embodiments, the inflatable patch does not house or contain a gas-generating formulation. In some embodiments, the inflatable patch does not include a water permeable surface. In some embodiments, the inflatable patch may be shaped and sized for contacting a partial inner circumference of the intestine.

In various embodiments, a compressed-state auto-inflatable patch assembly comprises an auxiliary gas-generating reaction chamber positioned on the outer surface of the compressed-state auto-inflatable patch.

In another aspect, there is provided auto-inflatable patch assembly comprising: a pliable and inflatable patch having water and gas-impermeable walls (“inflatable patch walls”), which define an internal chamber; one or more auxiliary gas-generating reaction chambers distinguished from and fluidly connected to the inflatable patch, said reaction chamber defined by pliable reaction chamber walls that house a first and second reactant in a single compartment, said first and second reactants configured to effervesce when exposed to water (e.g., in gas, liquid or vapor form); said reaction chamber walls being configured to constrain gas (i.e., substantially gas impermeable) and having at least a portion being a water-permeable outer surface.

In various embodiments, a single compartment may be separated from the inflatable patch by a formulation-retentive element. In some embodiments of the present aspect, the patch assembly further comprises an array of penetrating needles disposed on the surface of the inflatable patch.

The water-sensitive gas-generating formulation typically includes a first reactant being a water-soluble organic acid (e.g., citric acid) and a second reactant being an inorganic salt (e.g., alkaline carbonate, alkaline bicarbonate). In various embodiments of the present aspect, the water-sensitive gas-generating formulation may be configured for extended release. For example, it may include a viscosity enhancer. In another example, the water-sensitive gas-generating formulation may include a disintegrant or superdisintegrant in an intragranular portion of the formulation. For example, the water-sensitive gas-generating formulation may include granules comprising the first reactant, second reactant and a disintegrant or superdisintegrant. In another example, the water-sensitive gas-generating formulation may include a viscosity enhancer which makes up an extra-granular portion. For example, a first water-soluble organic acid (e.g., citric acid), inorganic salt (e.g., alkaline carbonate, alkaline bicarbonate), and a viscosity enhancer may make up an extra-granular portion.

In various embodiments, the water-sensitive gas-generating formulation may be a single unit such as a solid dosage form (e.g., multi-layer tablet, bilayer tablet, minitablet, or micro tablet). In some embodiments, the amount of first and second reactant amount is less than 80% of the water-sensitive gas-generating formulation. In some embodiments, the first and second reactants are in anhydrous form. In some embodiments, the disintegrant is a sugar, e.g., sorbitol. In some embodiments, the gas-generating reaction chamber comprises two or more water-permeable surfaces, for example, two or more opposing water-permeable surfaces.

In a another aspect, there is provided a method for providing an auto-inflatable patch assembly for delivering a therapeutic agent into an intestinal wall of a subject comprising: orally administering to the subject a device comprising a swallowable enteric outer shell and an auto-inflatable patch assembly, said patch assembly comprising a pliable and inflatable patch having water and gas-impermeable walls (“inflatable patch walls”), which define an internal chamber and an array of penetrating needles disposed on a surface thereof, auxiliary gas-generating reaction chambers defined by pliable reaction chamber walls that house a water-sensitive gas-generating formulation; said reaction chamber walls being configured to constrain gas (i.e., substantially gas impermeable) and having at least a portion being a water-permeable outer surface; exposing a water-sensitive gas-generating formulation to water through a water-permeable outer surface of the gas-generating reaction chamber such that the formulation generates an extended release of gas that flows towards the inflatable patch while being retained within its respective auxiliary gas-generating reaction chamber; and optionally further reaching a gas equilibrium at a constant pressure for a period of time.

In another aspect, there if provided a method for making an auto-inflatable patch assembly for delivering a therapeutic agent into an intestinal wall of a subject comprising: forming an extended-release water-sensitive gas-generating formulation; providing an upper and lower water and gas-impermeable layer, said layer having a portion being a water-permeable surface; positioning a water-sensitive gas-generating formulation adjacent to the water-permeable surface and further providing a formulation-retentive element between the formulation and the inflatable patch such that each gas-generating formulation may be retained within its respective auxiliary gas-generating reaction chamber; scaling a seam connecting the upper and lower layers to form an auto-inflatable patch; folding or rolling the auto-inflatable patch assembly within a capsule wherein water-permeable surface may be maintained on an outer surface of the compressed state auto-inflatable patch.

### BRIEF DESCRIPTION OF THE FIGURES

Aspects of the present disclosure are best understood from the following detailed description when read with the accompanying figures. It is emphasized that, under standard practice in the industry, various features are not drawn to scale. The dimensions of the various features may be arbitrarily increased or reduced for clarity of discussion. In addition, the present disclosure may repeat reference numerals and/or letters in multiple examples.

FIG. 1 illustrates a perspective view of an expanded auto-inflatable patch assembly according to one or more embodiments.

FIG. 2A-C illustrates a schematic, view of the top, bottom, and cross-sectional view of an expanded, uninflated form of the auto-inflatable patch assembly (e.g., after the disintegration of the outer shell), according to one or more embodiments.

FIG. 3 illustrates a schematic view of multiple gas-generating chambers within the arca of the inflatable patch, according to one or more embodiments.

FIG. 4 illustrates a schematic view of an auto-inflatable patch assembly within a capsule, expanded state, and expanded inflated state.

FIG. 5 illustrates a schematic view of an auto-inflatable patch assembly in a human intestine in its compressed state according to one or more embodiments.

FIG. 6 illustrates a schematic view of an auto-inflatable drug delivery device for delivery of a therapeutic in a human intestine in its inflated state according to one or more embodiments.

FIG. 7A illustrates a schematic bottom view of an expanded inflated form of the patch assembly, according to one or more embodiments.

FIG. 7B illustrates a schematic top view of an expanded, uninflated form of the auto-inflatable patch, according to one or more embodiments.

FIG. 7C illustrates a lateral view of an expanded, uninflated form of the auto-inflatable patch assembly before the gas-generating formulation begins to disintegrate, according to one or more embodiments.

FIG. 8A illustrates a bottom view of an expanded, semi-inflated form of the auto-inflatable patch assembly after the reactants have begun the gas-generating reaction, according to one or more embodiments.

FIG. 8B illustrates a top view of an expanded, semi-inflated form of the auto-inflatable patch assembly after the reactants have begun the gas-generating reaction, according to one or more embodiments.

FIG. 8C illustrates a lateral view of an intestine containing an expanded, semi-inflated form of the auto-inflatable patch, according to one or more embodiments.

FIG. 9A illustrates a bottom view of an expanded, inflated form of the auto-inflatable patch assembly after the reactants have progressed with the gas-generating reaction, according to one or more embodiments.

FIG. 9B illustrates a top view of an expanded, inflated form of the auto-inflatable patch assembly after the reactants have progressed with the gas-generating reaction, according to one or more embodiments.

FIG. 9C illustrates a lateral view of an intestine containing an expanded, inflated form of the auto-inflatable patch assembly after the device is deployed and penetrating needles pierce the intestine lumen wall.

FIG. 10, 11, 12 are schematic views of multiple configurations of gas-generating chambers according to one or more embodiments.

FIG. 13 illustrates the position of the gas-generating chamber in the compressed state and one method of rolling and folding the auto-inflatable patch assembly device into a capsule, according to one or more embodiments.

### DETAILED DESCRIPTION OF THE INVENTION

In the following detailed description of the aspects of the invention, numerous specific details are outlined to provide a thorough understanding of the disclosed embodiments. However, it will be evident to one skilled in the art that the embodiments of this disclosure may be practiced without these specific details. In other instances, well-known methods, procedures, components, and circuits have not been described in detail so as not to unnecessarily obscure aspects of the embodiments of the invention. And, to avoid needless descriptive repetition, one or more components or actions described by one illustrative embodiment can be used or omitted as applicable from other exemplary embodiments.

The inventors have discovered an expansion force and force per area that is sufficient to deliver the penetrating needles to a relevant layer for the release of a therapeutic agent, while maintaining the gas-generating reaction chamber and/or inflatable patch seams intact. This force may be influenced by a combination of factors including amount of gas-generating formulation, volume of the internal volume of the auto-inflatable patch assembly as well as structural integrity of the walls of the patch assembly. In should be recognized that the forces per area may be approximately equivalent to the force per area applied to the base of the penetrating needles or array thereof. Thus, in another aspect, as well as any of embodiments of the auto-inflatable patch assembly described herein, the auto-inflatable patch assembly may be configured to provide a force per area of less than 1.0 N/cm². In some embodiments, the force per area may be less than 0.9 N/cm². In some embodiments, the force per area may be less than 0.8 N/cm². In some embodiments, the force per area may be less than 0.7 N/cm². In some embodiments, the force per area may be less than 0.5 N/cm². In some embodiments, the force per area may be less than 0.3 N/cm². In some embodiments, the force per area may be less than 0.1 N/cm². In some embodiments, the force per area may be more than 0.09 N/cm. In some embodiments, the force per area may be more than 0.15 N/cm². In some embodiments, the force per area may be more than 0.20 N/cm². In some embodiments, the force per arca may be more than 0.30 N/cm². In some embodiments, the force per area may be more than 0.40 N/cm². In some embodiments, the force per area may be more than 0.50 N/cm². In some embodiments, the force per area may be more than 0.50 N/cm². In any of the embodiments of the auto-inflatable patch assembly described herein, a sufficient amount of force per area may be more than 2 pound per square inch (psi) as measured at 37° C. In any of the embodiments of the auto-inflatable patch assembly described herein, a sufficient amount of force per arca may be more than 3 pound per square inch (psi) as measured at 37° C. In any of the embodiments of the auto-inflatable patch assembly described herein, a sufficient amount of force per arca may be more than 5 pound per square inch (psi) as measured at 37° C. In any of the embodiments of the auto-inflatable patch assembly described herein, a sufficient amount of force per arca may be less than 5 pound per square inch (psi) as measured at 37° C. In any of the embodiments of the auto-inflatable patch assembly described herein, a sufficient amount of force per arca may be less than 4 pound per square inch (psi) as measured at 37° C. In any of the embodiments of the auto-inflatable patch assembly described herein, a sufficient amount of force per arca may be between 1 and 5 pound per square inch (psi) as measured at 37° C. In any of the embodiments of the auto-inflatable patch assembly described herein, a sufficient amount of force per arca may be between 2 to 4 psi as measured at 37° C.

Embodiments of the invention provide devices, systems, and methods of use for an auto-inflatable patch assembly, with particular application in the body, especially in the intestine, which is adapted to inflate upon moisture, water, or fluid exposure. In the context of the intestine, the auto-inflatable patch assembly may inflate and subsequently contact and apply pressure to at least a portion of the inner circumference of the intestine. The auto-inflatable patch assembly includes a pliable and inflatable patch having water and gas-impermeable walls, which define an internal chamber and a plurality of penetrating needles disposed on a surface thereof. The inflatable patch may be fluidly connected to one or more auxiliary gas-generating reaction chambers, each housing a gas-generating formulation. Each gas-generating reaction chamber is defined by pliable reaction chamber walls that house a water-sensitive gas-generating formulation (e.g., extended-release formulation); said reaction chamber walls configured to constrain gas (i.e., substantially gas impermeable) and have at least a portion being a water-permeable outer surface.

The patch assembly may be configured such that each gas-generating formulation may be retained within its respective auxiliary gas-generating reaction chamber. In some embodiments, each gas-generating formulation is configured to contact an inner surface of the gas-generating reaction chamber. In some embodiments, the gas-generating formulation may be positioned and/or maintained adjacent (i.e., on the inner side of) the water-permeable surface. In some embodiments, the gas-generating formulation may be prevented from migrating from the water-permeable surface, for example, by a formulation-retentive element adapted to allow fluid gas flow while retaining the gas-generating formulation within the gas-generating reaction chamber. For example, formulation-retentive element could include a netted sac or filter dividing the gas-generating chamber from the inflatable patch such that the formulation may be retained within the gas-generating chamber while allowing free flow of gas. In some embodiments, no water or fluid may be contained within the auto-inflatable patch assembly to drive the effervescent reaction aside from that which was absorbed from the outside.

More specifically, embodiments of the invention provide devices, systems, and methods of use of an auto-inflatable patch assembly for delivery of a therapeutic agent into, or through, a wall of a lumen, for example, an intestine lumen wall wherein an auto-inflatable patch assembly includes penetrating needles on a surface of a water-impermeable inflatable patch, wherein the inflatable patch may be fluidly connected to one or more gas-generating reaction chambers which house a gas-generating formulation (e.g., an extended-release gas-generating formulation), said gas-generating reaction chambers having at least a portion of a surface being a water-permeable surface. Alternatively, the inflatable patch may be fluidly connected to one or more gas-generating reaction chambers which house a gas-generating formulation, wherein the gas-generating formulation may be positioned adjacent to an internal wall having a water-permeable surface.

As used herein, “expanded form” is meant to refer to a form which is not compressed, folded, or rolled for example and ready to be disposed within a swallowable enteric shell. The expanded form may be inflated or uninflated, depending on the context.

As used herein, “compressed form” is meant to refer to a state which is folded, rolled or otherwise dimensionally reduced to fit within a smaller container for example, a swallowable enteric shell.

As used herein, and unless otherwise specified, “gas impermeable” or “impermeable to gas” relates to a material, membrane, or wall that hinders gas permeation by delaying, preventing, or restricting the exit or diffusion of a substantial amount of gas across its surface for a certain period. Typically, a wall that is “gas impermeable” or “impermeable to gas” includes a closely packed network of molecules or polymers that restrict the diffusion of gas molecules across its surface, effectively hindering but not necessarily wholly preventing gas permeation. For example, the reaction chamber walls are configured to constrain gas, and the choice of material or membrane for the wall is guided by resistance however, gas permeability may exist and occur slowly or depend on a combination of factors including but not limited to material (e.g., polymer type) and width. In addition, although the reaction chamber wall is described as impermeable, this does not necessarily include the portion of the reaction chamber walls having a water-permeable outer surface. In some embodiments, the permeable outer surface is directionally permeable to water in any form, including gas or liquid form, and as such, is gas permeable. In addition, a skilled artisan will recognize that the qualities of a gas-impermeable membrane in the GI tract may change over time so while a reaction chamber wall is considered gas impermeable, this quality may change over time in vivo and is meant to relate to the characteristic at time=0. Once the portion having water permeability absorbs water, it changes its physical characteristics including but not limited to elasticity, thickness, gas permeability, etc.

As used herein, and unless otherwise specified, “impermeable to water” or a “water-impermeable” surface, wall, or membrane prevents the passage of water molecules due to its dense arrangement of polymer chains, forming a barrier that maintains a water-impermeable state.

As used herein, the two reactants, or a first and second reactant, are used interchangeably, and both interact as effervescent in the presence of water to release gas.

As used herein, “material” is a composition that makes up a portion, element, component, or the like.

As used herein, the expanded state is meant to relate to the state of the device outside of the enteric outer shell or in a state which is not uncompressed, folded, rolled or otherwise dimensionally reduced.

As used herein, “extended-release” or controlled release” is used interchangeably to relate to a formulation designed to gradually release for example, a gas, over an extended period of time compared to an equivalent immediate release.

The term “lumen” refers herein to the inside space of a tubular structure. Examples of lumens in a body include arteries, veins, and tubular cavities within organs. In all cases, the lumen should have the natural presence of water or fluid, or it should be possible to supply water or fluid to the lumen.

The term “lumen wall” refers to a wall of a lumen, where the wall includes all layers from an inner perimeter to an outer edge of the lumen, such as concerning lumens in a body, the mucosa, submucosa, muscularis, serosa, and an outer wall of the lumen, with the constituent blood vessels and tissues.

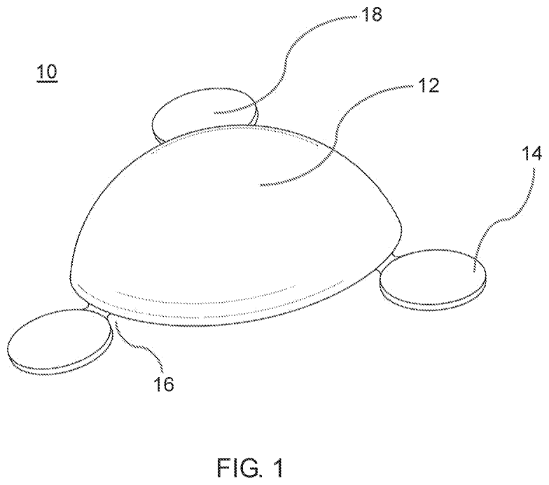

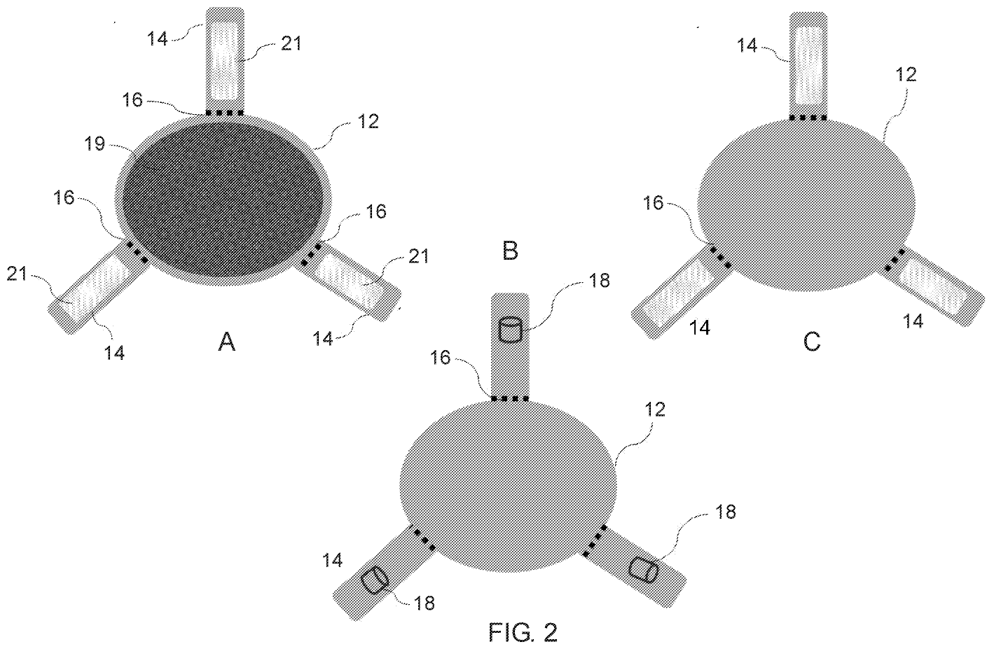

Referring to FIGS. 1 and 2, an inflatable patch assembly is illustrated. FIG. 1A and 1B, is a perspective view of an inflated auto-inflatable patch assembly 10 (herein “patch assembly”) according to one or more aspects is presented, while FIG. 2A, B and C illustrates a schematic, a top, bottom and cross-sectional view of an expanded, uninflated form of the auto-inflatable patch assembly 10 (i.e., after the disintegration of the outer shell), according to one or more embodiments. In FIG. 1, patch assembly 10 is illustrated in its expanded form after release from the swallowable enteric outer shell. Patch assembly 10 includes a pliable and inflatable patch 12 having gas and water-impermeable walls which define an internal chamber and a plurality of penetrating needles disposed on a surface thereof. The auxiliary gas-generating reaction chambers 14 are defined by pliable reaction chamber walls that house one or more water-sensitive gas-generating formulations 18, said reaction chamber walls being configured to constrain gas (i.e., substantially gas impermeable) and having at least a portion being a water-permeable outer surface. The patch assembly is configured such that each gas-generating formulation 18 or set of gas-generating formulations is retained within its respective auxiliary gas-generating reaction chamber 14 during inflation. For example, a formulation-retentive element 16 may employ size to restrict the passage of gas-generating formulation 18. Patch assembly 10 includes a formulation-retentive element 16 which divides between inflation chamber 12 and the reaction chamber 14. Inflation chamber 12 does not include a water permeable surface. The internal chambers of the inflation patch 12 and respective interiors of the one or more auxiliary gas reaction chambers 14 are configured to constrain gas within which ultimately causes inflation to occur. In some embodiments, despite an amount of pressure produced within gas-generating reaction chamber 14, substantial gas leakage from the auto-inflatable patch does not occur in a first period. That being said, as time progresses, and continued gas is generated while applying additional pressure to the walls, and pressure equilibrium is eventually reached. In a second period, gradual gas leakage may occur. In some embodiments, the gas leakage drives release of the auto-inflatable patch from the intestinal wall.

In some embodiments, the water-sensitive gas-generating formulation 18 is an extended-release gas-generating formulation. In some embodiments, the water-sensitive gas-generating formulation 18 is configured to generate gas to provide a pressure of the inflatable patch 12 as measured at 37° C. of more than 2, 3, or 3.5 psi. In some embodiments, water-sensitive gas-generating formulation 18 is configured to generate gas to provide a pressure of the inflatable patch 12 as measured at 37° C. of about 2 to 8, 2 to 6 or 2 to 5 psi. In some embodiments, water-sensitive gas-generating formulation 18 is configured to generate gas to provide a substantially consistent pressure of the inflatable patch as measured at 37° C. for about 30 minutes. In some embodiments, water-sensitive gas-generating formulation 18 is configured to generate gas to provide a substantially consistent pressure of the inflatable patch of more than 3.0 psi as measured at 37° C. for about 30 minutes. In some embodiments, extended-release gas-generating formulation 18 includes a viscosity enhancer. In some embodiments, extended-release gas-generating formulation 18 comprises a first and second reactant configured to effervesce in the presence of water. In some embodiments, the first and second reactant are housed within a single compartment.

Although three auxiliary gas-generating reaction chambers 14 are depicted in the FIG. 1, in some of the embodiments, any number of chambers may be provided. Inflatable patch 12 is separated from each gas-generating reaction chamber 14 by a formulation-retentive element 16. Formulation-retentive element 16 is adapted to retain each gas-generating formulation 18 within its respective auxiliary gas-generating reaction chamber 14 (i.e., prevented from migrating from the water-permeable surface or maintained adjacent to the water-permeable surface). Formulation-retentive element 16 (dotted line) is adapted to enable fluid-only flow, i.e., gas and water. In some embodiments, three auxiliary gas-generating reaction chambers 14 are preferred. In some embodiments, the auxiliary gas-generating reaction chambers 14 are spaced apart equally from one and other.

In some embodiments, a formulation-retentive element is positioned along an inflatable patch outer circumference, perimeter, outer or side surface. In some embodiments, two or more formulation-retentive elements are positioned along an outer circumference, perimeter, outer or side surface of the inflatable patch. In some embodiments, three or more formulation-retentive elements are positioned along an outer circumference, perimeter, outer or side surface of the inflatable patch. In some embodiments, two to five formulation-retentive elements are positioned along an outer circumference, perimeter, outer or side surface of the inflatable patch. In some embodiments, the water-sensitive gas-generating formulation 18 is an extended-release gas-generating formulation.

FIGS. 2A, B and C illustrate a schematic, view of top (A), bottom (C) and cross-sectional (B) view of an expanded, uninflated form of the auto-inflatable patch assembly 10 (e.g., after the disintegration of the outer shell), according to one or more embodiments. In top view (A), a plurality of penetrating needles 19 are disposed on a surface of the pliable and inflatable patch 12. Although the inflatable patch 12 and gas-generating reaction chamber 14 are configured to constrain gas therein (e.g., are gas-impermeable), a portion of gas-generating reaction chamber 14 is a water-permeable outer surface 21. In FIG. 2B, a cross-sectional view illustrates the auxiliary gas-generating reaction chambers 14 defined by pliable reaction chamber walls that house the water-sensitive gas-generating formulation 18 in the gas-generating reaction chambers 14 of the patch. Formulation-retentive element 16 separates between the inflatable patch 12 and the gas-generating reaction chambers 14. In the illustration, three are presented although, in embodiments, any number may be possible. In FIG. 2C, a bottom cross-sectional view is identical to the top cross-sectional however, a plurality of penetrating needles is not present. Rather, the bottom portion of the inflatable patch is configured to face the lumen or center of the intestine, rather than a wall.

FIGS. 2A, B and C may be used to illustrate the component for manufacture. In FIG. C, the bottom portion of the patch assembly may be prepared by providing a pliable, gas and water impermeable membrane, while cutting windows for a water permeable membrane. In some embodiments, at least 50% of the outer surface of the reaction chamber wall is water permeable. In some embodiments, at least a portion of the reaction chamber walls are water absorbing. And preferably directionally permeable to water-whether water in vapor or liquid form. In this case, the water-permeable surface of the chamber wall may have limited swelling such that the wall absorbs from 20% to 100% of the weight of dry resin as measured by the equilibrium water content. For example, the portion of the water permeable reaction chamber walls may be a hydrophilic membrane such as a hydrophilic elastomer, e.g., a hydrophilic polyurethane. The hydrophilic polyurethane may be a thermoplastic polyurethane.

Water-sensitive gas-generating formulation 18 may be prepared by granulation of the first and second reactant with a disintegrant (e.g., a super disintegrant) and subsequent combination with an extra granular viscosity agent. The final formulation may then be compressed into a tablet for example and placed on the bottom layer in the area adjacent to the water permeable membrane. The upper layer may be prepared in the same manner as the bottom layer, with the addition of an array of penetrating members containing a therapeutic agent on a surface of the inflatable patch. The two layers (both upper and lower) surrounding the tablets may then be welded along the perimeter and a formulation-retentive element 16 may be added for example by welding or fusing the upper and lower membranes.

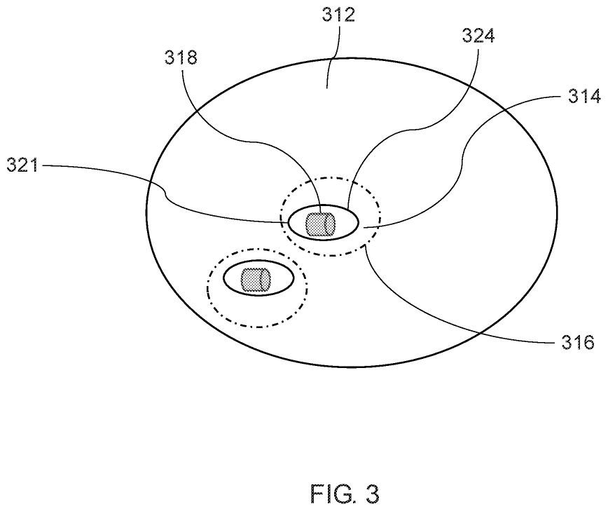

FIG. 3 illustrates an alternative configuration of the present invention wherein the plurality of gas-generating reaction chambers 314 are surrounded by the inflatable patch 312 and the formulation-retentive element 316 is positioned along an outer circumference, perimeter, outer or side surface of one or more gas-generating reaction chambers 314. In one embodiment, two gas-generating chambers 314, although more or less is also possible. Multiple gas-generating chambers 314 are dispersed within the general area of the inflatable patch 312, however, they are separated by the formulation-retentive element 316 which is configured to enable fluid passage while restricting passage of the water-sensitive gas-generating formulation 318. A water-absorbing portion 321 makes up a portion of gas-generating reaction chambers 314 and is bordered by a substantially gas-tight seam 324 where two materials, i.e., a water permeable and water impermeable membrane meet. The two materials may have substantially the same chemical make in order that they weld together hermitically. In some embodiments, a relatively gas-tight seam 324 connects any two of the following: a reaction chamber wall, an inflatable patch wall or a water-permeable outer surface of the reaction chamber wall. In some embodiments, a portion of a water-permeable outer surface of the reaction chamber wall meets a water impermeable wall at a relatively gas-tight seam 324.

A formulation-retentive element 316 is adapted to allow fluid gas flow while ensuring that the gas generation formulation 318 remains adjacent to the water-absorbing surface 321 (e.g., membrane or film).

A formulation-retentive element 316 is positioned between the inflatable patch 312 and the gas-generating reaction chamber 314. Formulation-retentive element 316 is adapted to enable the passage of gas while preventing the passage of the water-sensitive gas-generating formulation 318. In some embodiments, formulation-retentive element 316 is adapted to allow for directional gas flow of gas. In some embodiments, the directional flow occurs naturally based on the science of an equilibrium. In this way, the water transferred from the outside to the inside of reaction chamber 314 further interacts with the water-sensitive gas-generating formulation 318 to produce gas which drives the expansion of inflatable patch 312. Although the gas can freely pass through the formulation-retentive element, the formulation-retentive element does not enable passage of the water-sensitive gas-generating formulation 318, which continuously reacts to produce gas for a period of time. In some embodiments, formulation-retentive element 316 includes formulation-retentive element 316, adapted to maintain the gas-generating formulation 18 within the gas-generating reaction chamber 314.

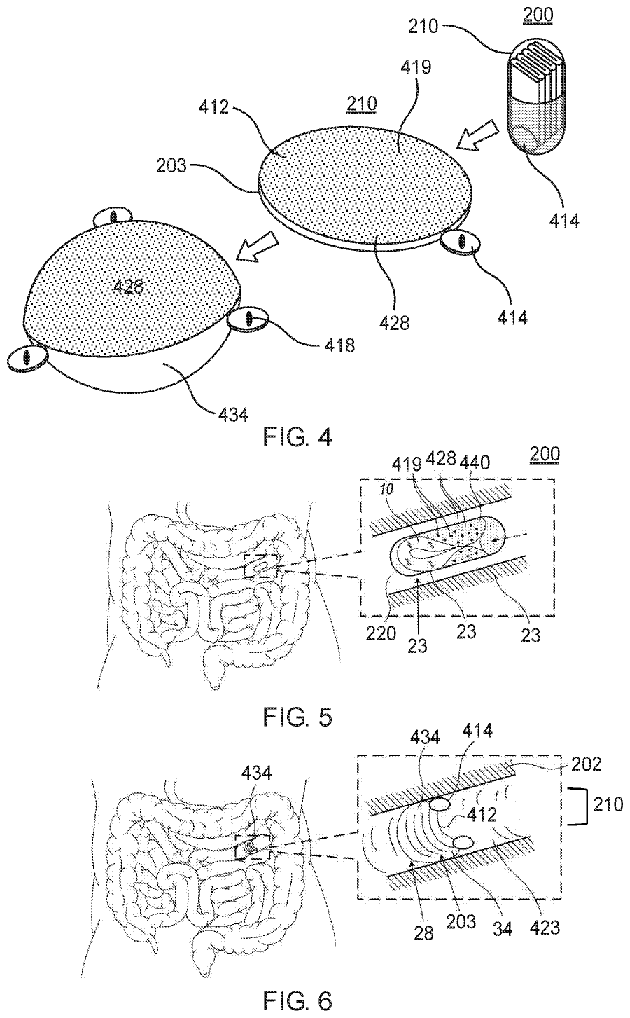

Referring now to FIGS. 4, 5, and 6, an auto-inflatable patch assembly 10 disposed within a swallowable enteric outer shell. FIG. 4 illustrates the sequence of events from prior to administration and until after administration to a subject. In one or more aspects of the present invention, the auto-inflatable patch assembly is contained within or configured to be contained within a swallowable enteric outer shell 220 (i.e., having pliable walls) by rolling or folding. For example, prior to administration, the auto-inflatable patch assembly is in a compressed (e.g., folded or rolled) state and disposed within a swallowable enteric outer shell. Note that the auxiliary gas-generating reaction chambers 414 are positioned on an outer portion of the compressed state. In this way, gas generation may occur immediately after a portion of the enteric outer shell has disintegrated and early gas generation may contribute to expansion by unfolding or unrolling.

The auto-inflatable patch assembly 410 further includes one or more gas-generating reaction chambers 414. Gas-generating reaction chamber 414 is defined by reaction chamber walls which define an internal volume and include a portion having water permeability. The gas-generating reaction chambers 414 also houses a water-sensitive gas-generating formulation 418 (e.g., solid dosage form) comprising and first and second reactants which effervesce in the presence of water to produce gas upon exposure to water molecules (liquid or vapor form). As used herein, a solid dosage form in the context of the water-sensitive gas-generating formulation is meant to include tablets such as compressed or molded. In some embodiments, powders and granules are excluded.

Although walls of both the inflatable patch 412 and gas-generating reaction chamber 414 are configured to constrain gas therein, i.e., being gas impermeable, the walls of the gas-generating reaction chamber 414 provide at least a portion of an outer surface that is configured to deliver water molecules therethrough, whether in liquid or solid form so that water on the outside or surface of the wall is transferred into the internal volume of the reaction chamber 414. In some embodiments, at least a portion of an outer surface is water absorbent. In some embodiments, the reaction chamber walls are water permeable. The auto-inflatable patch assembly 410 is configured such that in its compressed state, the gas-generating chamber is exposed on an outer surface. Although many different approaches to folding and rolling may be envisioned, at least one gas-generating chamber wall may be on an outer surface of the device's compressed or folded/rolled state such that it is exposed to water at an early timepoint.

In FIG. 5, the auto-inflatable patch assembly 200 comprises an outer shell 220 for example, a swallowable enteric outer shell 220. The outer shell 220 is configured to resist degradation in the gastric environment until arriving at the small intestine of the subject where it dissolves in the small intestine wall 202 in the presence of intestinal fluid to expose the auto-inflatable patch assembly 210. Typically, the outer shell 220 is pH-sensitive and may be configured to dissolve within 15 minutes (e.g., within 10 or 5 minutes). For some applications, auto-inflatable patch assembly 210 is biodegradable along the gastrointestinal tract. For some applications, auto-inflatable patch assembly 210 and all of its parts, comprise materials that either biodegrade or pass safely through the remaining gastrointestinal tract without sharp or rigid elements with the exception of the penetrating needles which are configured to pierce the tissue. Typically, the outer shell 220 has a length of at least 5 mm, no more than 30 mm, and/or between 5 and 30 mm. Typically, the outer shell 220 has a diameter of between 3 and 6 mm or between 4 and 5 mm. The auto-inflatable patch assembly 210 has a compressed shape when disposed within the outer shell 220. The auto-inflatable patch assembly 210 consists of the inflatable patch 412 having upper surface 428 and lower surface 434, each having an inflatable patch wall 203, which is gas and water impermeable and includes penetrating needles 419 on an upper surface 428 configured to face the intestinal lumen wall 423 in expanded form prior to penetration. Once expanded, due to a consistent amount of gas generation to apply a sufficient degree of pressure, the upper surface 428 of an inflatable patch 412 establishes contact with the intestinal wall 423 and remains until the penetration needles 428 have deposited the therapeutic agent. Typically, one or more penetrating needles 419 penetrate intestinal wall 423 to release the active agent without releasing therapeutic agent in the lumen itself.

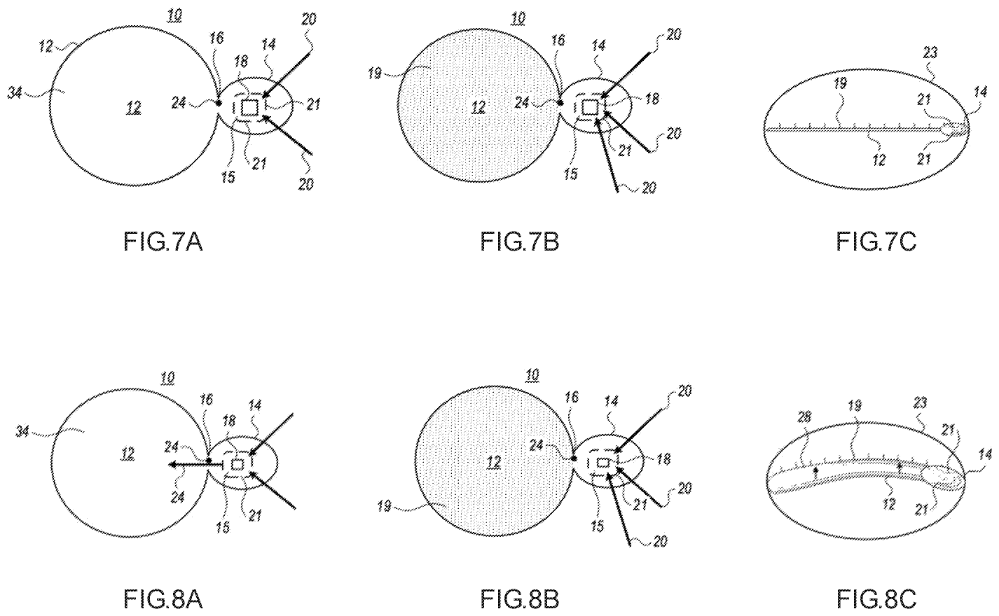

Inflatable patch 412 is operably connected to the gas-generating reaction chamber 414, which is folded such that it forms an outer surface of the compressed auto-inflatable patch assembly 210 immediately under the outer shell 220, as illustrated in FIG. 4. In this way, once the outer shell 220 begins to degrade, the gas-generating chambers 414 can begin exposure to intestinal fluid and initiate gas generation through chemical reaction between the two reactants, as will be discussed in FIG. 7A, 7B, and 7C. The shape and dimensions of inflatable patch 412 in its gas expanded state are such that it does not contact a full perimeter of the intestinal lumen wall 423 of small intestine wall 202. Auto-inflatable patch assembly 200 typically remain axially stationary in the small intestine for a release period to enable the release of an active agent while the penetrating needles 419 are within the intestinal wall 202, even under contraction of the small intestine wall. Following delivery of the active agent, the auto-inflatable patch assembly 210 continues through the GI and is eventually passed from the body. The effectivity of the inflatable patch 212 is generally unaffected by peristalsis (muscle contraction within the intestine) and does not block liquids or food passing through the intestine.

Further in FIG. 6, upon disintegration of the enteric outer shell, the auto-inflatable patch assembly is in expanded but uninflated or partially inflated state. The walls of inflatable patch 412 are pliable and substantially impermeable to gas and water. Inflatable patch 412 is configured to inflate upon receiving gas within the internal volume or from the adjacent gas-generating reaction chamber 414. No portion of inflatable patch 412 is adapted to absorb water.

Referring now to FIGS. 7 to 9, auto-inflatable patch assembly 10 includes a surface which is fluid sensitive and configured to inflate after absorbing water (or vapor) 20 (i.e., on a surface of the gas-generating reaction chamber) from the surrounding area, such as the intestinal tract. Absorption of water in the proximity of the water-sensitive gas-generating formulation causes the formulation to generate gas 22 and subsequently cause directional gas flow 30 from the gas-generating chamber towards the empty inflatable patch through a fluid-only passage having a formulation-retentive element 16 configured to maintain the formulation in the gas-generating reaction chamber. In some embodiments, the inflatable patch 12 further include penetrating needles 19 configured to deliver a therapeutic when gas pressure is applied to deliver one or more penetrating needles 19 to and through the surface of a lumen wall upon expanding the inflatable patch 12.

The gas-driven inflation mechanism is illustrated in detail. In FIG. 7A, 7B and 7C, the outer shell 220 has disintegrated within the small intestine lumen to provide an exposed auto-inflatable patch assembly 10. The auto-inflatable patch assembly 10 is a GI fluid automated drug delivery device that can deliver one or more therapeutic agents in solid or fluid form upon an inflatable patch's expansion. The exposed auto-inflatable patch assembly 10 assumes an unconstrained shape and the auto-inflatable patch assembly 10 expands, such as by unfolding and/or unrolling, in response to no longer being constrained by the outer shell 220 e.g., a coating or a capsule, a coating over a capsule, or a capsule over a coating).

The auto-inflatable patch assembly 10 includes inflatable patch 12 and gas-generating reaction chamber 14, which houses the gas-generating reaction chamber. A therapeutic agent in the solid formulation may make up a portion of the penetrating needle 19 on the surface (solid within penetrating needle) of the inflatable patch. Alternatively, a therapeutic agent may be in fluid form housed within (liquid) the inflatable patch 12. The auto-inflatable patch assembly 10 may include an inflatable patch 12, a gas-generating chamber 14 having a seam surrounding a window having a water absorbing surface 15, and housing a gas-generating formulation 18, a formulation-retentive element 16 such that the gas is fluidly connected between the gas-generating formulation 18 and the inflatable patch 12, and formulation-retentive element. Note that along the formulation-retentive element, which connects the gas-generating chamber 14 to the inflatable patch 12, a gas-tight seam exists at the transition between the water-permeable surface 15 and the remainder of chamber 14. The gas-generating reaction chamber 14, which is now exposed to the intestinal fluid and includes a surface which is water or specifically vapor permeable 15 bordered by seam and water and gas impermeable frame, begins to absorb water, moisture or vapor from the intestinal fluid which activates the gas-generating formulation 18 housed within a generating reaction chamber 14. Although the illustration shows a circular chamber wall surrounding the formulation, other shapes are also possible, such as a square or rectangular shape.

In FIG. 8A, 8B and 8C, and a reaction between the two reactants contained within the gas-generating formulation 18 begins to generate gas, eventually causing a gas flow towards the inflatable patch 12, which drives inflation although there is no increase in the size of a first and second major axis (the circle diameter in the Figure) of the inflatable patch 12, FIG. 7C. Illustrates the thickening of the patch inflatable patch 12. This inflation drives deployment at the intestinal lumen wall 23, illustrated in FIG. 7C. Inflatable patch 12 has an upper (intestinal-wall-contact) surface 28 and a lower (intestinal-lumen-facing) surface 34, which face in generally opposite directions and a plurality or an array of penetrating needles (e.g., microneedles) 19 operably connected or disposed on the upper surface 28 which is pliable. The gas-generating chamber 14, having at least a surface being water or vapor permeable and having an osmotic force inward towards the internal volume of the gas-generating chamber 14, continues to absorb water (e.g., vapor), resulting in sufficient gas generation to flow through the formulation-retentive element 16 and continue to inflate and apply pressure to the penetration members 26 which are configured with a tip to pierce the intestinal lumen wall 23. The shape and size characteristics of the gas-generating formulation enable it is retained by a retaining feature within the gas-generating chamber, while the retaining feature allows the free flow of gas towards the inflatable patch.

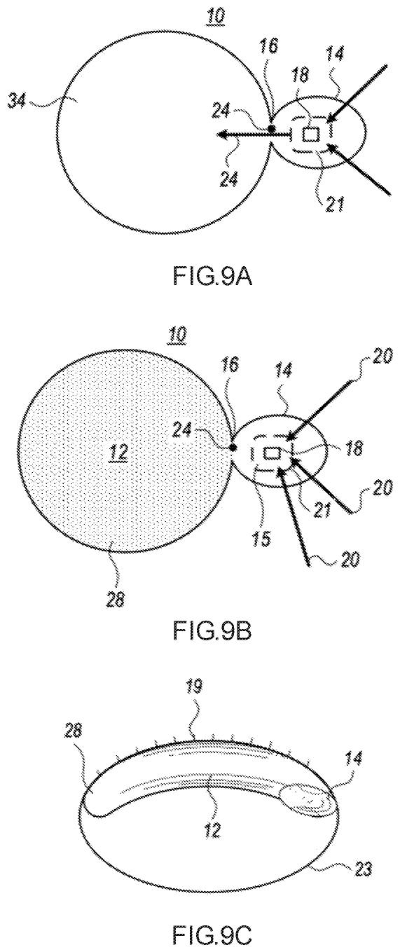

In FIG. 9A, 9B and 9C, even in the fully expanded form of the auto-inflatable patch assembly 10, the inner perimeter of the small intestine is not in full contact with the inflatable patch 12 (i.e. it does not encompass the total internal volume of the lumen at any specific location), such that food and liquids may pass through to the remainder of the small intestine, as illustrated in FIG. 8C. In addition, the force being applied to the wall is generated from inside the device without an opposing lumen wall force. Typically, an inflatable patch 12 has an elastomeric character, such as a plastic material (e.g., thermoplastic polyurethane or silicone). Preferably, the surface of the inflatable patch 12 does not stretch and forms a substantially flat surface such that it has a variance of height of about 5 mm or 4 mm or 3 mm or 2 mm.

In one aspect, there is provided an auto-inflatable patch assembly comprising: an inflatable patch having inflatable patch walls which define an internal volume, said walls being pliable and impermeable to gas and water; three or more gas-generating reaction chamber, each chamber fluidly connected to the inflatable patch via a fluid-only passage. The gas-generating reaction chamber may be defined by reaction chamber walls which house an internal volume and house a water-sensitive gas-generating formulation (e.g., solid dosage form or single unit) comprising two reactants; said reaction chamber walls being configured to constrain gas (e.g., having a degree of gas impermeability and having at least a portion of an outer surface being water-permeable or configured to deliver water molecules therethrough. In some embodiments, the gas-generating reaction chamber may be a single compartment that houses two reactants. In some embodiments, the gas-generating reaction chamber includes at least a portion of a water-permeable outer surface. For example, a portion may consist of at least 50%. In some embodiments, the gas-generating reaction chamber comprises two or more opposing water-permeable surfaces. In some embodiments, the gas-generating reaction chamber may be positioned along an outer circumference, perimeter, outer or side surface of the inflatable patch. In some embodiments, the auto-inflatable includes two or more gas-generating reaction chambers. In some embodiments, the auto-inflatable contains three or more gas-generating reaction chambers. In some embodiments, the auto-inflatable includes three gas-generating reaction chambers. In some embodiments, the auto-inflatable includes four gas-generating reaction chambers. In some embodiments, the water-sensitive gas-generating formulation may be a solid dosage form. In some embodiments, the water-sensitive gas-generating formulation may be a single unit. In some embodiments, the auto-inflatable patch assembly may be configured to minimize substantial gas leakage from the collective internal volume. In some embodiments, the pressure produced within the gas-generating reaction chamber does not cause substantial gas leakage from the auto-inflatable patch. That being said, in some embodiments, the gas-generating reaction chamber may be configured to purposely release gas after a period of time for example, via the water-permeable outer surface. In some embodiments, the gas-generating reaction chamber may be configured to release gas as pressure increases or reaches an equilibrium. Thus, in some embodiments, the auto-inflatable patch assembly may be configured, after a period of time, to decrease in pressure to subsequently disengage from a lumen wall such as the intestinal wall.

In another aspect, there is provided an auto-inflatable patch assembly comprising: an inflatable patch having inflatable patch walls which define an internal volume, said walls being pliable and impermeable to gas and water; one or more gas-generating reaction chambers fluidly connected to the inflatable patch however, being separated by a formulation-retentive element, said chamber defined by reaction chamber walls which houses a single compartment containing two reactants which generate gas upon exposure to water; said reaction chamber walls being configured to constrain gas therein (e.g., gas impermeable) and having at least a portion of an outer surface configured to deliver water molecules therethrough (e.g., being water-permeable); and a formulation-retentive element positioned between the inflatable patch and the gas-generating reaction chamber. In some embodiments, the gas-generating reaction chamber may be a single compartment that houses two reactants. In some embodiments, the gas-generating reaction chamber includes at least a portion of an outer surface that may be water-permeable. For example, a portion may include at least 50%. In some embodiments, the gas-generating reaction chamber comprises two or more opposing water-permeable surfaces. In some embodiments, the gas-generating reaction chamber may be positioned along an outer circumference, perimeter, or side surface of the inflatable patch. In some embodiments, the auto-inflatable includes two or more gas-generating reaction chambers. In some embodiments, the auto-inflatable includes three or more gas-generating reaction chambers. In some embodiments, the auto-inflatable includes three gas-generating reaction chambers. In some embodiments, the auto-inflatable includes four gas-generating reaction chambers. In some embodiments, the water-sensitive gas-generating formulation may be a solid dosage form. In some embodiments, the water-sensitive gas-generating formulation may be a single unit. In some embodiments, the auto-inflatable patch assembly may be configured to minimize substantial gas leakage from the collective internal volume.

In another aspect, there is provided an auto-inflatable patch assembly comprising: an inflatable patch having inflatable patch walls which define an internal volume, said walls being pliable and impermeable to gas and water; one or more gas-generating reaction chambers fluidly connected to the inflatable patch via a fluid-only passage, said chamber defined by reaction chamber walls which house an internal volume and a water-sensitive gas-generating formulation, said walls having at least 50% of an outer wall surface being a water-permeable surface; and a formulation-retentive element positioned between the inflatable patch and the gas-generating reaction chambers adapted to enable passage of gas and prevent passage of the water-sensitive gas-generating formulation. In some embodiments, the gas-generating reaction chamber may be a single compartment that houses two reactants. In some embodiments, the gas-generating reaction chamber includes at least a portion of an outer surface that may be water-permeable. For example, a portion may include at least 50%. In some embodiments, the gas-generating reaction chamber comprises two or more opposing water-permeable surfaces. In some embodiments, the gas-generating reaction chamber may be positioned along an outer circumference, perimeter, outer or side surface of the inflatable patch. In some embodiments, the auto-inflatable includes two or more gas-generating reaction chambers. In some embodiments, the auto-inflatable includes three or more gas-generating reaction chambers. In some embodiments, the auto-inflatable includes three gas-generating reaction chambers. In some embodiments, the auto-inflatable includes four gas-generating reaction chambers. In some embodiments, the water-sensitive gas-generating formulation may be a solid dosage form. In some embodiments, the water-sensitive gas-generating formulation may be a single unit. In some embodiments, the auto-inflatable patch assembly may be configured to minimize substantial gas leakage from the collective internal volume.

In another aspect, there may be provided an auto-inflatable patch assembly comprising: an auto-inflatable patch assembly comprising: an inflatable patch having inflatable patch walls which define an internal volume, said walls being pliable and impermeable to gas and water; a gas-generating reaction chamber fluidly connected to the inflatable patch via a formulation-retentive element, said chamber defined by walls which define an internal volume housing a water-sensitive gas-generating formulation comprising two reactants, said walls having at least a portion of a wall being water-permeable; wherein the inflatable patch walls, chamber walls and connections thereof are adapted to minimize substantial gas leakage from the internal volume. In some embodiments, the device includes a formulation-retentive element positioned between the inflatable patch and the gas-generating reaction chamber, which may be adapted to enable fluid-only passage and prevent the water-sensitive gas-generating formulation. In some embodiments, the gas-generating reaction chamber may be a single compartment which houses two reactants. In some embodiments, the gas-generating reaction chamber includes at least a portion of a water-permeable outer surface. For example, a portion may include at least 50%. In some embodiments, the gas-generating reaction chamber comprises two or more opposing water-permeable surfaces. In some embodiments, the gas-generating reaction chamber may be positioned along an outer circumference, perimeter, outer or side surface of the inflatable patch. In some embodiments, the auto-inflatable includes two or more gas-generating reaction chambers. In some embodiments, the auto-inflatable includes three or more gas-generating reaction chambers. In some embodiments, the auto-inflatable includes three gas-generating reaction chambers. In some embodiments, the auto-inflatable includes four gas-generating reaction chambers. In some embodiments, the water-sensitive gas-generating formulation may be a solid dosage form. In some embodiments, the water-sensitive gas-generating formulation may be a single unit. In some embodiments, the auto-inflatable patch assembly may be configured to minimize substantial gas leakage from the collective internal volume. In some embodiments, the auto-inflatable patch assembly may be configured to minimize substantial gas leakage from the collective internal volume for a first time period. while providing gas release from the collective internal volume during a subsequent time period. This subsequent time period is after the portion of the water absorbing surface has absorbed an amount of water. This process is based on the change in characteristics which ensue during water absorption process.

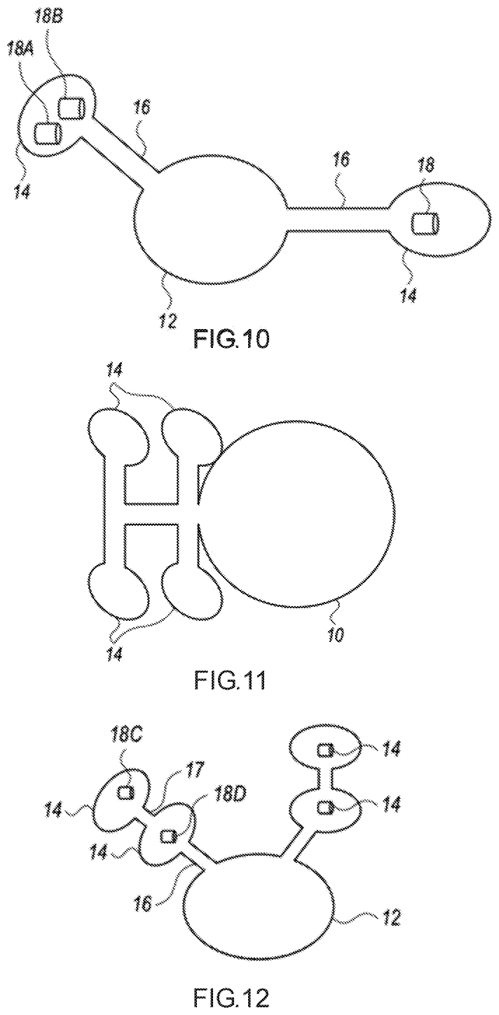

In FIGS. 10-12, multiple configurations for one or more gas-generating reaction chambers 14, inflatable patches 12, formulation-retentive element and gas-generating reaction formulations 18, are disclosed, although additional configurations can also be envisioned. In some embodiments, such as FIGS. 10, 11, and 12, a gas-generating reaction chamber extends outwards from a central inflatable patch. In some embodiments, the auto-inflatable patch assembly may include one or more gas-generating reaction chambers positioned on a circumference, perimeter, outer or side surface of the inflatable patch. In some embodiments described herein, the auto-inflatable patch assembly may include two or more gas-generating reaction chambers extending radially from an inflatable patch.

In any embodiments described herein, the auto-inflatable patch assembly may include an array of gas-generating reaction chambers. In some embodiments, the auto-inflatable patch assembly comprises about 2 to 5 or 2 to 4 gas-generating reaction chambers. The number of gas-generating reaction chambers may be selected to ensure that the chamber and patch remain intact despite the gas pressure build-up in the shared internal volume of the inflatable patch and gas-generating chamber. Including several gas-generating chambers enables a more gradual force over time, which contributes to the structural integrity of the patch.

For example, in FIG. 10, two gas-generating chambers 14 are illustrated, each gas-generating chamber 14 operably connected to the inflatable patch 12 by formulation-retentive element 16. Note that along the formulation-retentive element, which separates the gas-generating chamber 14 to the inflatable patch 12, a gas-tight seam exists at the transition between the water-permeable surface 15 and the remainder of chamber 14. According to the inventors, one of the challenges in obtaining an effective gas-generating system with minimum gas leakage despite the increasing pressure may be identifying material compatible with welding at the seams where a transition occurs.

In yet another embodiment, FIGS. 11 and 12 illustrate gas-generating members arranged in a series (see, for example, 14) or parallel (see for example, 14). It should be understood that any constellation of gas-generating chambers can be contemplated as exemplified by the figures, including multiple gas-generating formulations positioned adjacent to a water-absorbent membrane said formulation or wall and contained within the internal volume of the gas-generating chamber by a formulation-retentive element may be contemplated.

In FIG. 11, various single and multiple-unit gas-generating formulations 780 are also illustrated. The first and second reactants may be formulated in single or multiple units within the internal volume of the gas-generating chamber 14. As shown in FIG. 11, the gas-generating formulation 18 comprises two portions, separated into different compartments of the gas-generating chamber. The first portion housed a first compartment having gas-generating formulation 18A, and the second portion housed in a second compartment having gas-generating formulation 18A. Each compartment may be separated by a conduit 16. One portion (formulation 18A) may contain a first reactant dependent on intestinal fluid exposure for disintegration, such as potassium bicarbonate, and the other portions (formulation 18B) includes a second reactant, such as citric acid dependent on intestinal fluid exposure for disintegration which reacts with the disintegrated first reactant to produce a gas such as CO2. This may add a degree of stability during shelf life when there may be an expectation that water vapor from humidity may affect the functionality of the reaction.

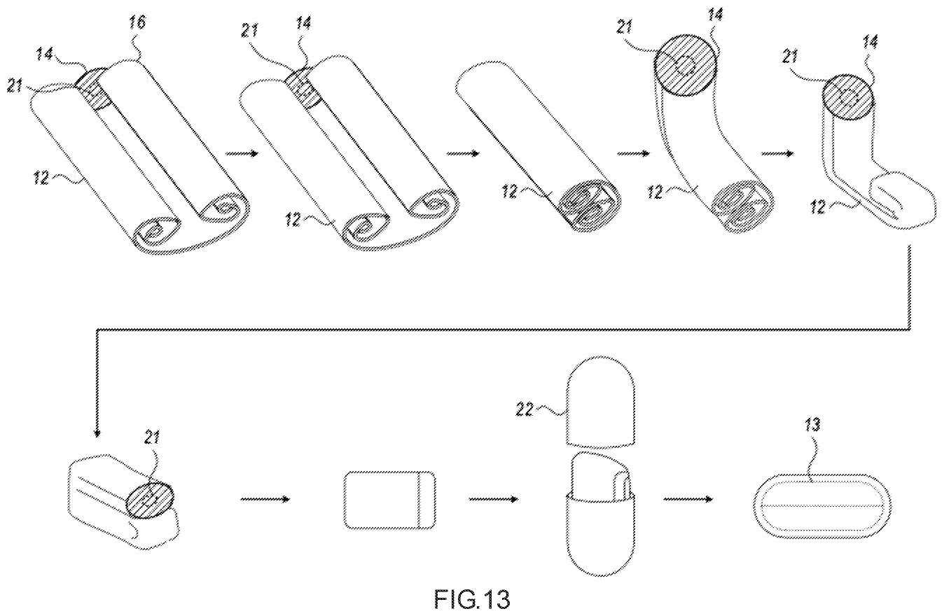

In various embodiments of the auto-inflatable patch assembly described herein, the auto-inflatable patch assembly includes an inflatable patch. In some embodiments, the inflatable patch walls may be defined by walls that define an internal volume (e.g., a maximum internal volume). In some embodiments, the inflatable patch may be configured to receive a fluid (e.g., gas) and maintain the fluid therein. In some embodiments, the inflatable patch walls may be pliable. The term pliable relates to a flexible or easily bent, folded or rolled quality, as illustrated in FIG. 13 where the position of the gas-generating chamber in the compressed state and one method of rolling and folding the auto-inflatable patch assembly device into a capsule, according to one or more embodiments.

In some embodiments, the inflatable patch walls may be impermeable to gas and water. In some embodiments, the inflatable patch includes inflatable patch walls which define an internal volume, said walls being pliable and impermeable to gas and water. The inflatable patch may configure to prevent water ingress while also preventing gas leakage from the internal volume. In some embodiments, the inflatable patch includes no portion which may be capable of absorbing water. Said another way, no walls of the inflatable patch has water-absorbing properties. In some embodiments, the inflatable patch may be configured to constrain gas generated by the gas-generating reaction chamber. In some embodiments, constraining gas received from the gas-generating reaction chamber can be based on shape and/or material, as discussed below. In some embodiments, the gas-generating reaction chamber and/or gas-generating formulation are contained outside an internal volume of the inflatable patch.

In some embodiments, the inflatable patch walls have limited elasticity or stretching. In some embodiments, the inflatable patch walls are characterized by being made from material that does not easily change its shape or expand when subjected to pressure or force. Said another way, the wall undergoes deformation or expansion based on its pliability and in response to applied pressure or volume change. For example, the inflatable patch walls may comprise a polymer, a substantially non-compliant polymer, polyethylene, PET or polyimide.

As used herein, the first and second major axis is the largest and second largest dimension of the inflatable patch. Typically, when the inflatable patch expands from a compressed state to a gas-inflated state, there is a minimum change in the size of the first and second major axis. Typically, only a unidirectional expansion in the third major axis i.e., the patch width, increases in size, as illustrated in FIGS. 7C, 8C, and 9C.

In various embodiments of the auto-inflatable patch assembly described herein, the auto-inflatable patch assembly may be employed for drug delivery. In this case, the inflatable patch may be pliable to transition from a compressed to an expanded state and vice versa. In some embodiments, the compressed state may be folded or rolled to be further housed within a swallowable outer shell. In the folded or rolled state, the gas-generating reaction chambers may be positioned on an outer surface to be exposed to fluid and drive transition into an expanded state due to gas generation. Before adding the outer shell, the inflatable patch may be folded and/or rolled to a suitable size (e.g., a size suitable for disposable in a 00 or 000-size capsule). In some embodiments, the inflatable patch may be folded or rolled such that upon capsule disintegration (or at least partial disintegration), the inflatable patch may be only partially exposed to GI fluid.

In some embodiments, the gas inflation drives unfolding or unrolling and positioning adjacent to the inner circumference of the small intestine lumen and subsequent penetration of the penetrating needles. This limited elasticity or stretching may play a role in ensuring that the gas being generated can apply a pressure (e.g., gradual and extended pressure) to the intestine's surface rather than continually stretch until the inflatable patch erupts.

In some embodiments, the inflatable patch may have at least one surface operably connected to one or more tissue-penetrating needles configured to deliver a therapeutic agent through the intestine wall. As used herein, through an intestine wall is meant to relate to piercing or penetration of the intestinal tissue for delivery within the wall layers. Typically, the inflatable patch is not connected to the outer shell in any manner. Ideally, the outer shell ideally is substantially no longer intact when the inflatable patch begins to inflate. The penetrating needles are configured to deliver therapeutic agent in liquid or solid form. When providing a liquid form of therapeutic agent, the penetrating needles act to deliver the liquid of a therapeutic agent from a therapeutic agent reservoir. When providing a solid form of therapeutic agent, the penetrating needles can be a solid formulation of therapeutic agent, including for example a biodegradable microneedle, for disintegration, dissolution or disengagement and release in the intestine tissue, i.e., in the intestine tissue of the intestine wall.

In some embodiments, the inflatable patch comprises a portion of a surface operably connected to an array of penetrating needles configured to deliver therapeutic agents. In some embodiments, the inflatable patch includes an array of about 20 to about 250 penetrating needles per cm².

In some embodiments, the inflatable patch may be shaped to include two relatively flat or planar surfaces facing opposite directions.

In some embodiments, the inflatable patch does not include a gas-generating first or second reactant within its internal volume.

In some embodiments, the inflatable patch may have a cross-sectional circle shape (e.g., disk shape). However, additional shapes are possible, including, but not limited to, a cross-sectional square, rectangle, triangle, etc. The cross-sectional shape may include rounded or sharp edge corners. In some examples, the inflatable patch may be shaped as a circle with two relatively flat surfaces. In some embodiments, the inflatable patch has an upper and lower surface facing in opposite directions wherein the lower may be configured to open in an intestinal-lumen-facing manner, and the upper may be configured to open in an intestinal lumen wall-facing manner.

In some embodiments, the inflatable patch may be configured to be a gas-expanded inflatable patch sized to contact only a portion of an inner circumference of an intestine wall. Said another way, the expanded state of the inflatable patch may be sized to enable passage of GI fluid or other GI contents. Thus, in some embodiments, the inflatable patch may be configured to apply a gas-driven force to an array of needles. In some embodiments, the inflatable patch may be configured to apply a gas-driven force to an array of needles without a contributing pressure provided by an opposing intestinal wall. Thus, in some embodiments, the gas pressurizes the inflatable patch and expands the inflatable patch to an inflated state. The inflatable patch may be shaped with an upper and lower surface such that pressure within the inflatable patch aligns an upper surface of the inflatable patch against tissue at a delivery site. In this manner, orientation of the patch occurs.

In some embodiments, the inflatable patch may be inflated by gas pressure which drives the expansion of the inflatable patch wall of the patch into a gas-inflated state. In other embodiments, the inflatable patch may be configured to expand from a compressed state to an expanded state by gas. In some embodiments, the inflatable patch may be fluidly connected to one or more gas-generating reaction chambers directly or via a fluid-only passage. As used herein, a fluid connection enables fluid (e.g., gas) flow from the gas-generating reaction chamber or chambers thereof and into the inflatable patch by a gas gradient at initial stages of gas generation however, eventually, the gas flow reaches an equilibrium and gas pressure may be maintained.

In some embodiments, the inflatable patch may be fluidly connected to an adjacent but distinct gas-generating reaction chamber positioned outside and has distinct qualities from the inflatable patch. In some embodiments, the inflatable patch may be fluidly connected to one or more gas-generating reaction chambers via a fluid-only passage.

In some embodiments, the inflatable patch may be configured to connect fluidly with an array of fluid-only passages, each fluidly connected to a gas-generating reaction chamber. In some embodiments, the inflatable patch may be fluidly connected to more than two gas-generating reaction chambers via a fluid-only passage per chamber. In some embodiments, the inflatable patch may be fluidly connected to three gas-generating reaction chambers via a fluid-only passage per chamber. For clarity, despite the fluid connection, the inflatable patch and the auxiliary gas-generating reaction chamber are distinct in being separated by a formulation retention element, nature of the surface, having a portion of a surface being water permeable in the case of the reaction chamber, and in terms of housing the gas-generating formulation.

In some embodiments, the inflatable patch includes a substantially flat surface. For example, in some embodiments, the inflatable patch includes a first and second major surface, preferably of similar or equivalent size. In any of the embodiments of the auto-inflatable patch assembly described herein, the inflatable patch includes a substantially flat surface even in the expanded state. In this way, the flat surface can be configured to press against the intestinal wall (IW) upon expansion of the inflatable patch in a relatively even manner, thereby applying a consistent force on the array of penetrating needles. In some examples, the array of penetrating needles may be positioned in a constant direction on a single plane. In some examples, the array of penetrating needles faces opposing directions.

In any of the embodiments of the auto-inflatable patch assembly described herein, the inflatable patch has an upper and lower surface that face in generally opposite directions and where the upper surface may be configured to open (from the compressed to the expanded state) in an intestine wall facing manner. In any of the embodiments of the auto-inflatable patch assembly described herein, the inflatable patch has an upper and lower surface such that, upon inflation, contacts a portion of an inner circumference of the intestine (e.g., small intestine) to allow contact with the intestine lumen wall but enable solid, or fluid passage through an intestinal lumen. In this way, instead of an inflatable increasing such that the volume of the inflatable patch blocks a whole cross section of the lumen, a portion of the intestinal lumen may be open for passage of food and liquid. Thus, the inflatable patch attaches to the intestine lumen wall surface without blocking food and liquid. In any of the embodiments of the auto-inflatable patch assembly described herein, the inflatable patch may be sized to accommodate a variety of interpopulation anatomies such as due to contact of only a partial inner circumference of the intestine wall. In any embodiments of the auto-inflatable patch assembly described herein, the inflatable patch may be configured to accommodate a variety of intestinal contraction states or peristatic forces, such as due to contact of only a partial inner circumference of the intestine wall.

In any of the embodiments of the auto-inflatable patch assembly described herein, the inflatable patch applies an internally generated force to penetrate through the intestine wall without dependence on a contributing power provided by an opposing intestinal wall.

In any of the embodiments, the inflatable patch has a maximum dimension in the direction of the major axis that may be less than about 9 cm, less than 8 cm, less than 7 cm, less than about 6 cm, less than about 5.5 cm, or less than about 5 cm.

In any embodiments, the inflatable patch may be shaped to form an internal volume. In any embodiments, the inflatable patch may be shaped to form an internal volume of less than 30 mL. In any of the embodiments of the auto-inflatable patch assembly described herein, the inflatable patch may be shaped to form an internal volume of less than 20 mL. In any embodiments of the auto-inflatable patch assembly described herein, the inflatable patch may be shaped to form an internal volume of less than 10 mL. In any of the embodiments of the auto-inflatable patch assembly described herein, the inflatable patch may be shaped to form an internal volume of less than 8 mL. In any of the embodiments of the auto-inflatable patch assembly described herein, the inflatable patch may be shaped to form an internal volume between 4 and 10 mL. In any of the embodiments of the auto-inflatable patch assembly described herein, the inflatable patch may be shaped to form an internal volume of between 5and 10 mL. In any of the embodiments of the auto-inflatable patch assembly described herein, the inflatable patch may be shaped to form an internal volume of between 6 and 10 mL. In any of the embodiments of the auto-inflatable patch assembly described herein, the inflatable patch may be shaped to form an internal volume of between 4 and 9 mL. In any of the embodiments of the auto-inflatable patch assembly described herein, the inflatable patch may be shaped to form an internal volume of between 5 and 9 mL. In any of the embodiments of the auto-inflatable patch assembly described herein, the inflatable patch may be shaped to form an internal volume of between 5 and 8 mL. In any of the embodiments of the auto-inflatable patch assembly described herein, the inflatable patch may be shaped to form an internal volume of between 5 and 7 mL.

In any of the embodiments of the auto-inflatable patch assembly described herein, the inflatable patch includes an upper surface having multiple layers. A portion of the inflatable patch (e.g., the upper surface) may be configured to have pressure equalizer properties to improved contact between the inflatable patch surface and the intestinal wall so as deliver the therapeutic agent (e.g., tissue penetrating needles) over a selected area in an even manner. In any of the embodiments of the auto-inflatable patch assembly described herein, the inflatable patch has multiple layers including a first layer and a second layer which are fixed together at fixation locations. In any of the embodiments of the auto-inflatable patch assembly described herein, the inflatable patch has one or more layers to provide a structure that minimizes or prevents stretching of another layer or other layers. For example, the inflatable patch may include a first layer of an elastic polymer, a second layer of nylon mesh, and a third layer of an clastic polymer may be present.

In any of the embodiments of the auto-inflatable patch assembly described herein, the inflatable patch may be substantially impermeable to liquid or gas. It should be understood that the term impermeable in this context may be relative and changing due to the changing environment and especially due to gradual gas pressure being applied to the inflatable patch. For example, there may be slow release of gas from the inflatable patch once the gas pressure reaches a certain threshold. That being said, the system depends on the inflatable patch constraining the gas to obtain the desired shape and volume to deploy adjacent to the intestinal lumen wall and further drive the penetrating needles into the intestinal wall. For example, wherein the inflatable patch may be shaped and sized for contacting a partial inner circumference of the intestine.

Various materials are described herein as relevant for fabricating the inflatable patch. In any embodiments, the inflatable patch includes a water-impermeable surface and does not have a portion having a water-absorbing surface. For example, the inflatable patch may a polymer such as a thermoplastic elastomer (TPE), which may, for example, comprise a thermoplastic polyurethane (TPU); polyurethane (PU) or polyethylene (PE), which are substantially impermeable to liquid or gas. More specifically, examples include but are not limited to, a poly(ether urethane) selected from TECO flex® EG-80A, Tecoflex® EG-85A, TECO flex® EG-93A, or Chrono Thane™ T75A, T75B, T75C or T75D polyurethane. In some examples, a coating on the outer surface of the inflatable patch may be included. The membrane thickness may be less than 40 cm, less than 30 cm or less than 400 μm. The membrane thickness may be less than 300 μm. The membrane thickness may be less than 200 μm. In some examples, the inflatable patch further includes a delayed or gradually dissolving deflation valve configured to release gas at the end of the period of time. Alternatively, the gas-generating reaction is complete, and no further gas is generated.

Examples of penetration members suitable for the present disclosure are described in U.S. patent application Ser. No. 17/562,899, entitled Controlled release formulations and methods of targeted drug delivery within the small intestine, and filed December 27, 2021, the disclosure of which is hereby incorporated by reference in its entirety.

In any of the embodiments of the auto-inflatable patch assembly described herein, the auto-inflatable patch assembly include at least one penetrating needle. In any of the embodiments, the auto-inflatable patch assembly include an array of penetrating needles of about 50 to 250 penetrating needles per cm². In any of the embodiments, the auto-inflatable patch assembly include an array of penetrating needles of about 100 to 150 penetrating needles per cm². In some embodiments, the tissue penetrating needle has a base configured to receive a force and a penetrating tip. In some embodiments, the tissue penetrating needle has sufficient hardness or mechanical strength to be advanced into the intestinal wall by applying a force to the tissue penetrating needle or base thereof. In some embodiments, the tissue penetrating needle may be an elongated member having a base at one end and a tapered, sharpened, or honed tip at one end thereof, which may be straight or curved. Other shapes are contemplated, including a pyramid or cone.

As used herein, “penetrating” is meant to include any disruption for example, in at least one region of a mucosa or submucosa or muscularis mucosae barrier in the small intestine after being intralumenally deployed in the subject.

In any of the embodiments of the auto-inflatable patch assembly described herein, the auto-inflatable patch assembly includes tissue penetrating needles having a length of between 0.02 and 6.0 mm or between 0.5 and 2.8 mm. In some embodiments, the penetrating needles extend from a surface of an inflatable patch and have a length of between 1.4 and 2.8 mm. In some embodiments, the penetrating needles extend from a surface of an inflatable patch and have a length of between 1.4 and 2.0 mm. In some embodiments, the penetrating needles extending from a surface of an inflatable patch have a length of between 1.4 and 1.7 mm. In other embodiments, the tissue penetrating needles may have a length of about 20 and 1000 μm.

For some applications, the average density of all medication needles on the surface to which medication needles are attached may be between 2 and 6 needles/mm2, such as 4 needles/mm2. Alternatively or additionally, for some applications, each of the medication needles 1250 may be located within 1 mm of at least one adjacent medication needle.

In some embodiments, the tissue penetrating needle has sufficient stiffness to be advanced into the intestinal wall (i.e., soft tissue) by applying a force to the tissue penetrating needle or base. In any embodiments of the auto-inflatable patch assembly described herein, at least a portion of the penetrating needles are detachably coupled to the inflatable patch. In any embodiments of the auto-inflatable patch assembly described herein, at least a portion of the penetrating needles are configured to penetrate through the mucosa, submucosa, musculara, or serosa.

The auto-inflatable patch assembly may be inflated by gas produced within an auxiliary gas-generating chamber when the gas-generating formulation and specifically the first reactant and second reactant, are exposed to water (e.g., intestinal fluid) via a water-permeable surface. The water may be in any form, whether vapor or liquid form. In some embodiments, no water or fluid is present in the gas-generating chamber nor the inflatable patch unless of course it entered by way of the water permeable surface which can undergo swelling. In some embodiments, the auxiliary gas-generating reaction chamber may be defined by pliable reaction chamber walls that house a water-sensitive gas-generating formulation; said reaction chamber walls being gas impermeable and having at least a portion being a water-permeable outer surface. In this context, the water-permeable outer surface may be adapted to pass water molecules in a gas or liquid state. In some embodiments, although the wall having a water-permeable outer surface may be less than fully gas impermeable, the wall may be adapted to constrain gas despite the pressure increase within the internal chamber.

In some embodiments, the pliable reaction chamber walls are adapted to be rolled or folded into a compressed state for case of packaging within a swallowable enteric outer shell. In some embodiments, an auxiliary gas-generating reaction chamber may be positioned on an outer surface of the compressed state auto-inflatable patch.

The auxiliary gas-generating chamber may be fluidly connected to the internal chamber of the inflatable patch while being separated or divided by a formulation-retentive element. The formulation-retentive element may be configured to retain the formulation within its auxiliary gas-generating reaction chamber. In this way, the formulation-retentive element prevents migrating from the water-permeable surface and/or maintains the formulation adjacent to the water-permeable surface.

In some embodiments, the formulation-retentive element functions as a fluid passage, having a structural element or feature that selectively permits fluid passage or flow of fluid (e.g., liquid or gas including humidity), while effectively preventing the passage of solid particles. This is achieved through a design characterized by, for example, controlled pore size, which collectively facilitates the passage of fluid while entrapping or obstructing solid entities (e.g., of a minimum size). In some embodiments, the formulation-retentive fluid passage may be a conduit or channel defined within a structure engineered to allow unimpeded flow of fluids based on its low viscosity. The formulation-retentive fluid passage may be designed with dimensions that prevent the passage of solid particles above a certain threshold, thereby ensuring selective permeability that facilitates the transmission of fluids while effectively blocking the passage of solid entities due to geometric constraints.

In some embodiments, one or more auxiliary gas-generating reaction chambers are fluidly connected to the inflatable patch via a fluid-only passage or a formulation-retentive element. The chambers are defined by chamber walls which house an internal volume and a water-sensitive gas-generating formulation comprising a first and second reactant. In some embodiments, the gas-generating reaction chamber defines a single compartment or chamber housing both the first and the second reactant. In some embodiments, the single compartment does not contain a fluid or liquid. The two reactants combine with water to generate gas. The reaction chamber walls are, on one hand, gas impermeable or configured to constrain gas therein and on the other hand, at least partially water-permeable to deliver water molecules, whether liquid or vapor, therethrough. In some embodiments, the gas-generating reaction chamber may be configured to be compressed and expanded (e.g., by rolling or folding). In some embodiments, a gas-generating reaction chamber remains intact on the surface of the compressed patch assembly. In some embodiments, the chamber walls include at least 50%, at least 60% or at least 70% of an outer wall surface being a water-permeable surface. In some embodiments, a gas-generating reaction chamber comprises two or more opposing water-permeable surfaces.

In some embodiments, the gas-generating reaction chamber may be positioned along an outer circumference, perimeter, or side surface of the inflatable patch. For example, two or more gas-generating reaction chambers may be positioned along an outer circumference, perimeter, or side surface of the inflatable patch. In another example, three or more gas-generating reaction chambers are positioned along an outer circumference, perimeter, outer or side surface of the inflatable patch. In another example, two to five gas-generating reaction chambers are positioned along an outer circumference, perimeter, outer or side surface of the inflatable patch.

In some embodiments, the gas-generating reaction chambers may have a surface area of more than 800, 900, 1000, 1100 or 1200 mm². In other embodiments, the gas-generating reaction chambers may have a surface area of between 800 to 1800 mm². In other embodiments, the gas-generating reaction chambers may have a surface area of between 1200 to 1350 mm².

In any of the embodiments described herein, the plurality of gas-generating reaction chambers are configured to provide varying initiation times and/or rates of gas production. For example, the chamber may be coated with a layer that delays the initiation of water absorption and therefore gas generation. Alternatively, each chamber may house gas-generating formulations formulated to release gas at a different time and/or rate.

In any of the embodiments described herein, one or more gas-generating reaction chambers are configured to generate gas such that the patch may be deployed at and/or attached to an intestine wall within about 10 min.

In some embodiments, one or more gas-generating reaction chambers include a fluid-only passage disposed between the gas-generating reaction chamber and an inflatable patch to facilitate a flow path for gas between an internal volume of the gas-generating reaction chamber and the internal volume of one or more inflatable patches.

One of the challenges encountered during development, according to the inventors, was finding relevant biocompatible material for the fabrication of both the water-absorbent surface or membrane and the water-impermeable surface or membrane, each meeting a very different set of requirements for function, however without resulting in a gas leakage at the seam (i.e. welded seam), despite the evolving stress (increase in inner gas pressure) and presence in the intestinal lumen where fluids are present. This transition can be positioned radially between the gas-generating reaction chamber and the inflatable patch. Thus, in any of the embodiments of the auto-inflatable drug delivery devices described herein, the fluid-only passage includes a seam that is configured to minimize gas leakage. In some embodiments, the seam is configured to maintain integrity despite the pressure applied. Thus, in any embodiments of the auto-inflatable drug delivery devices described herein, the fluid-only passage includes a seam, for example, by hot melt, of two compatible membranes.

In any embodiments described herein, one or more gas-generating reaction chambers include a water-absorbent surface. In some embodiments, gas leakage may be minimized due to a relatively gas-tight seam on the border between the water-absorbent membrane and the water-impermeable membrane. In some embodiments, gas leakage may be minimized due to a relatively gas-tight seam between a water-absorbent window and the water-impermeable frame surrounding said window.

In some embodiments, a gas-generating reaction chamber may be configured to provide sufficient gas to the inflatable patch for a period of time sufficient to advance at least one tissue penetrating needle through an intestine wall.

Although effervescent formulations are known in the art, a shelf-stable water-sensitive gas-generating formulation adjacent to a water-absorbent surface presents unique challenges.

In any embodiments described herein, the auto-inflatable drug delivery device includes two or more gas-generating reaction chambers.

In any embodiments described herein, the auto-inflatable drug delivery device includes a gas-generating chamber configured to produce a consistent amount of gas over an extended period. For example, the patch assembly may reach an equilibrium between the gas generated and the gas escaping such that the gas pressure may be maintained.

In any embodiments described herein, the auto-inflatable drug delivery device includes a gas-generating chamber configured to produce a consistent amount of gas over an extended period of more than 30 minutes.

In any of the embodiments described herein, the auto-inflatable drug delivery device includes a gas-generating chamber having a thickness of between 15 and 100 or about 20 to 90 or about 20 to 80 or about 25-80 μm. In any of the embodiments described herein, the auto-inflatable drug delivery device includes a gas-generating chamber may have a surface area of more than 350 mm², or between 350 and 550 mm² or about 400 to 500 mm.²

Any elastic and biocompatible material can be used in the fabrication of the chamber wall. In some examples, the material may be non-resorbable. In some examples, the material may be resorbable. In any of the embodiments described herein, the chamber wall may be fabricated or coated to promote adhesion between the gas-generating formulation and the chamber wall.

The shape of the chamber can be any of a variety of shapes and configurations, so long as it provides a closed shape to contain the gas produced with a constricted lumen for exit.

In any of the embodiments of the auto-inflatable patch assembly described herein includes a gas-generating reaction chamber having a chamber wall. In any of the embodiments of the auto-inflatable patch assembly described herein includes a gas-generating reaction chamber fluidly connected to the inflatable patch through a fluid-only passage. As used herein, the “fluidly connected” in this context is meant to include a fluid pathway which allows free flow of gas within the internal volume of the gas-generating chamber towards the inflatable patch. In any of the embodiments of the auto-inflatable patch assembly described herein includes a gas-generating reaction chamber wherein no surface of the gas-generating reaction chamber may be connected to a tissue penetrating needle.

In any of the embodiments of the auto-inflatable patch assembly described herein, the gas-generating reaction chamber wall may be configured to limit the extent of swelling while facilitating a driving force for transport of water preferably water vapor. In any of the embodiments of the auto-inflatable patch assembly described herein, the gas-generating reaction chamber wall defines an internal volume which houses gas. In any of the embodiments of the auto-inflatable patch assembly described herein, the gas-generating reaction chamber wall may be made of material configured to be substantially gas impermeable.

In some embodiments, the invention provides auto-inflatable patch assembly includes at least a portion of a chamber wall configured facilitate transport of water. Transport of water can be liquid or gaseous (vapor) form of water. In preferably embodiments, the transport of water may be in gaseous (vapor) form. The water is typically readily available for absorption in the intestinal fluid once the outer shell may be at least partially broken down. Although the membrane may be somewhat gas impermeable, absorption of water provides a gelling effect to may provide for qualities different from the starting membrane such that the polarity of a gas such as water vapor, permits an extent of transfer through the membrane.

In some embodiments, the invention provides auto-inflatable patch assembly wherein at least a portion of the chamber wall may be a water absorbent or hydrophilic surface. In preferred embodiments, the chamber wall further includes a water and gas impermeable surface.

In any of embodiments of the auto-inflatable patch assembly described herein, the chamber wall may be configured to define an internal volume which houses and constrains gas, said chamber wall configured to be water absorbent while limiting the extent and rate of swelling.

In any of embodiments of the auto-inflatable patch assembly described herein, the chamber wall thickness may be less than 100 μm, less than 80, less than 70, less than 60 or less than 50 μm. In some embodiments, the chamber wall thickness may be between 20 and 100 um. In some embodiments, the chamber wall thickness may be between 25 and 80 μm. In some embodiments, the chamber wall thickness may be between 25 and 50 μm.

In any of embodiments of the auto-inflatable patch assembly described herein; the chamber wall has a surface area of between 300 to 600 mm². In some embodiments, the chamber wall has a surface area of between 400 to 500 mm².

In any of embodiments of the auto-inflatable patch assembly described herein, the chamber wall thickness promotes passing through the membrane of water in vapor or liquid form. In some embodiments, the chamber wall thickness allows for water in gas form to pass through the membrane. In some embodiments, the chamber wall thickness allows for water in liquid form to pass through the membrane.

In any of embodiments of the auto-inflatable patch assembly described herein, the chamber wall may be configured to constrain such that gas can be produced and constrained to form an outward gas flow from the chamber over a period of time towards the target inflatable patch. Typically, this would be optimized with selection of optimal shape and size to form a chamber or housing as well as relevant material which constrains gas.

In preferred embodiments of the auto-inflatable patch assembly described herein, the chamber wall can include any one or more materials, membranes or films which promote any one and preferably more than one and most preferably all of the below features: high flex and abrasion resistance to contain gas and drive flow of gas; resistant to dissolving in intestinal fluid over a period of time; pliable; biocompatibility and biostability; and compatible with other plastics to provide a gas-tight seam with the fluid-only passage or inflatable wall.

In some embodiments of the auto-inflatable patch assembly described herein; the chamber wall can include a hydrophilic membrane. As used herein, hydrophilic membranes arc nonporous films that breath via osmotic potential by absorbing and desorbing the vapor molecules and then transport the molecules to the over side. The water vapor molecules first adsorb on the outer surface of the chamber wall facing the intestinal lumen which has a higher vapor concentration than the inside of the chamber. The vapor molecules occupy the free volume between the polymer molecule chains and move across the membrane without interacting chemically with the polymer, thus maintaining the polymer intact and largely unaffected. Essentially, the amorphous regions act like intermolecular pores allowing water vapor to pass through but substantially prevent the penetration of liquid water. Upon arriving on the other side, they are desorbed into the surrounding internal volume of the gas-generating reaction chamber for interaction with the gas-generating formulation. Note that conventional polymers and rubbers typically used in inflatables do not have the polar groups required for activating the hydrophilic mechanism for transport of water. Some hydrophilic polymers which be considered such as e.g., poly(vinyl alcohol) (PVA), polyvinyl pyrrolidone, or the like and polyethylene oxide (PEO), would dissolve in water or swell such that flex and abrasion resistance would be poor.

In any of embodiments of the auto-inflatable patch assembly described herein; the chamber wall includes a hydrophilic membrane having a Shore hardness of from 70A to 65D. In preferred embodiments, the hydrophilic membrane has a Shore hardness of about 83A.

In any of embodiments of the auto-inflatable patch assembly described herein, the chamber wall the hydrophilic membrane may be a hydrophilic elastomer.

In any of the embodiments of the auto-inflatable patch assembly described herein, the chamber wall may be a hydrophilic membrane having a limited extent of swelling such that the material absorbs equilibrium water content from 20% to 100% of the weight of dry resin as measured by the equilibrium water content.

In any of embodiments of the auto-inflatable patch assembly described herein, the chamber wall the hydrophilic membrane may be fabricated from a hydrophilic elastomer. In preferred embodiments, hydrophilic elastomer may be at least partially made of a thermoplastic polyurethane, hydrophilic polyesters, or hydrophilic polyamides.

In any of embodiments of the auto-inflatable patch assembly described herein, the chamber wall the hydrophilic elastomer has a limited the extent of swelling such that the material absorbs equilibrium water content from 20% to 100% of the weight of dry resin. In some embodiments, the hydrophilic elastomer may be a multiblock poly(ether urethane) or a silicone poly(ether urethane). In some embodiments, the hydrophilic elastomer may be a multiblock poly(ether urethane) or a silicone poly(ether urethane). In some embodiments, the hydrophilic elastomer may be a multiblock poly(ether urethane). In some embodiments, the multiblock poly(ether urethane) may be water-swellable and comprises poly(ethylene oxide).

In any of the embodiments of the auto-inflatable patch assembly described herein, the chamber wall may be a thermoplastic polyurethane with an aliphatic, hydrophilic polyether-based resin (i.e., in the backbone of the polyurethane) such as ionomeric groups such as, but not limited to, carboxylic acids or urea which promote water absorption in the elastomer). Particular preferred hydrophilic polyurethanes are those which absorb water but swell much less, to the extent of about 20-100%, upon contact with an aqueous medium. In some examples, there are provided commercially available thermoplastic polyurethanes having hydrophilicity suitable for the present invention including but not limited to: such as Tecophilic™ thermoplastic polyurethane such as Tecophilic® HP-60D-20. Tecophilic® HP-60D-35, Tecophilic® HP-60D-60, or Tecophilic® HP-93A-100 (Lubrizol Advanced Materials, Inc.); Hydro Thane™ thermoplastic polyurethane (AdvanSource Biomaterials Corp.); Quadraphilic™ thermoplastic polyurethane (Biomerics, LLC); HydroMed™ (AdvanSource Biomaterials Corp.); or Dryflex® (HEXPOL TPE); or PurSil® (Pak Chromical Ltd.). It should be recognized that a variety of different polymers such as polyethylene oxide can be modified with enough hydrophilic groups like to increase their hydrophilicity and allow them to absorb water so it can be delivered through the chamber membrane or wall.

In any of the embodiments of the auto-inflatable patch assembly described herein; the thermoplastic polyurethane has a Shore durometer value from about 70A to about 65D. The particular material and its thickness and wall area can be selected to achieve the particular water absorption profile, i.e., water permeation rates.

As used herein, a seam may be used to describe a boundary of two membranes, films, or materials. In this context the membranes, films or materials may be the same or different. In some examples, different materials making up a seam provide an additional challenge in ensuring a hermetic (i.e., substantially gas-tight) seal.

One of the challenges in developing the present invention relates to the requirement of a hermetic (i.e., airtight) seam even under increasing gas pressure driven by the gas-generating reaction chamber. Thus, in any of embodiments of the auto-inflatable patch assembly described herein, the chamber wall as well as the gas-generating reaction chamber are configured to include substantially hermetic seams that ensures gas pressure formulation within the internal volume of the gas-generating reaction chamber as well as the inflatable compartment. In any of embodiments of the auto-inflatable patch assembly described herein, the inflatable patch and/or the gas-generating reaction chamber may be configured to prevent substantial gas leakage. This may be by employing for example compatible material and applying a specific heat to weld the seam.

In any of embodiments of the auto-inflatable patch assembly described herein, the chamber wall includes a first major surface and a second major surface which are substantially symmetrical. In any of embodiments of the auto-inflatable patch assembly described herein, the gas-generating reaction chambers have a substantially flat shape with a first major surface and a second major surface, each having a water impermeable frame portion with a water permeable window in the center of the frame. Typically, different materials are employed for the frame and the window portion and therefore a window seam is used to describe herein the boundary of the window or interface of the two membranes, films, or materials. Thus, in any of embodiments of the auto-inflatable patch assembly described herein, the window seam is configured to minimize or prevent substantial gas leakage from the auto-inflatable patch assembly.

In any of embodiments of the auto-inflatable patch assembly described herein, the gas-generating reaction chamber of formulation contained therein, is configured to generate a controlled amount of pressure to minimize substantial gas leakage from the auto-inflatable patch assembly. For example, the choice of materials, amount of gas and/or pressure applied to walls by gas from the gas-generating reaction chamber does not cause opening of a seam.

The number of gas-generating chambers in the auto-inflatable patch assembly described herein, may also be selected in order to minimize risk of gas leakage at a seam. In any of embodiments of the auto-inflatable patch assembly described herein, 2 or more gas-generating reaction chamber are present. In any of embodiments of the auto-inflatable patch assembly described herein, 3 or more gas-generating reaction chamber are present. In any of embodiments of the auto-inflatable patch assembly described herein; 2 to 6 gas-generating reaction chambers are present.

In any of the embodiments of the auto-inflatable patch assembly described herein, the chamber wall may be additionally coated or mixed with additional excipients to promote stability or adjust water absorption properties. Stability in this case is meant to refer to stable performance in the GI track despite to exposure to water in liquid and vapor form prior to exposure to water in the GI track such as during shelf life or manufacture.

In any of the embodiments of the auto-inflatable patch assembly described herein; the chamber wall compatible at a relatively gas-tight seam at the transition to the gas-generating reaction chamber wall.

In another aspect, there is provided, an extended-release gas-generating chamber for extended gas generation in the presence of water, said chamber defined by pliable reaction chamber walls that house in a single compartment a water-sensitive gas-generating formulation comprising a first and second reactant which effervesce in the presence of water; said reaction chamber walls being gas impermeable and having at least a portion being a water-permeable outer surface.

In some embodiments, the extended-release gas-generating chamber includes first and second reactants are in a single unit. In some embodiments, the extended-release gas-generating chamber includes an extended-release gas-generating formulation.

In some embodiments, the extended-release gas-generating chamber include a water-sensitive gas-generating formulation configured to generate gas to provide a pressure of the inflatable patch as measured at 37° C. of more than 2, 3, 3.5, 5, 7, or 10 psi. In some embodiments, the extended-release gas-generating chamber includes a water-sensitive gas-generating formulation configured to generate gas to provide a pressure of the inflatable patch as measured at 37° C. of more than 2, 3, or 3.5 psi. In some embodiments, the extended-release gas-generating chamber includes the water-sensitive gas-generating formulation configured to generate gas to provide a pressure of the inflatable patch as measured at 37° C. of about 2 to 8, 2 to 6 or 2 to 5 psi. In some embodiments, the extended-release gas-generating chamber includes a water-sensitive gas-generating formulation configured to generate gas to provide a substantially consistent pressure of the inflatable patch as measured at 37° C. for about 30 minutes.

In some embodiments, the extended-release gas-generating chamber includes an extended-release gas-generating formulation having a viscosity enhancer. In some embodiments, the single unit may be a solid dosage form, e.g., a multi-layer tablet, bilayer tablet, minitablet, or micro tablet. In some embodiments, the amount of first and second reactant amount may be less than 80% of the water-sensitive gas-generating formulation. In some embodiments, the first reactant may be a water-soluble organic acid (e.g., citric acid) and the second reactant may be an inorganic salt (e.g., alkaline carbonate, alkaline bicarbonate). In some embodiments, the extended-release gas-generating chamber includes a water-sensitive gas-generating formulation further comprises a disintegrating agent and a viscosity enhancer.

In some embodiments, the extended-release gas-generating chamber wherein the water-soluble organic acid (e.g., citric acid) and/or inorganic salt (e.g., alkaline carbonate, alkaline bicarbonate) are in anhydrous form. In some embodiments, the water-soluble organic acid (e.g., citric acid). an inorganic salt (e.g., alkaline carbonate, alkaline bicarbonate), and disintegrant make up granules. In some embodiments, the first reactant may be a water-soluble organic acid (e.g., citric acid) and the second reactant may be an inorganic salt (e.g., alkaline carbonate, alkaline bicarbonate). In some embodiments, the water-sensitive gas-generating formulation comprises granules including the first reactant, second reactant and a disintegrant or a superdisintegrant. In some embodiments, the water-sensitive gas-generating formulation comprises a viscosity enhancer which makes up an extra-granular portion. In some embodiments, water-soluble organic acid (e.g., citric acid), inorganic salt (e.g., alkaline carbonate, alkaline bicarbonate)), and a viscosity enhancer make up an extra-granular portion.

In some embodiments, the extended-release gas-generating chamber includes a gas-generating reaction chamber comprising two or more water-permeable surfaces. In some embodiments, the extended-release gas-generating chamber comprises two or more opposing water-permeable surfaces.

In some of the embodiments described herein, the auto-inflatable patch assembly includes a water-sensitive, gas-generating formulation disposed within the one or more gas-generating reaction chambers. As used herein, reference to a gas-generating formulation means a water-sensitive gas-generating formulation that includes two reactants which in the presence of water react to produce a gas. Thus, in any of the embodiments described herein, the auto-inflatable patch assembly includes a gas-generating formulation configured to generate gas upon exposure to water (in liquid or vapor form). In any of the embodiments described herein, the gas-generating formulation includes a first and second reactant. In any of the embodiments described herein, the first and second reactants are configured to produce gas upon exposure to water in liquid or gas form. In any of the embodiments described herein, the gas-generating formulation includes first reactant being an organic acid component and a second reactant being an carbonate salt component.

In some embodiments, the first and second reactants may be housed within a single compartment. In some embodiments, the water-sensitive gas-generating formulation may be a single unit comprising two reactants. In some embodiments, the water-sensitive gas-generating formulation may be a solid dosage form. For example, the solid dosage form may be a multi-layer tablet, bilayer tablet, minitablet or micro tablet.

In any of the embodiments described herein, the organic acid component may be a citric acid, malic acid, tartaric acid, ascorbic acid, fumaric acid, adipic acid, sodium hydrogen sulfate, succinic anhydride, monosodium phosphate (NaH₂PO₄), disodium diphosphate (NaH₂P₂O₇) and mixtures thereof. In any of the embodiments described herein, the carbonate salt component may be a sodium carbonate (NaHCO3), sodium bicarbonate (Na₂CO₃), sodium calcium bicarbonate (Ca(HCO3)2), sodium citrate, potassium carbonate, potassium bicarbonate, magnesium carbonate, calcium carbonate (CaCO3), and mixtures thereof. In any of the embodiments described herein, the carbonate salt may be a sodium bicarbonate. In some embodiments, the organic acid component may be a citric acid.

In some embodiments, the water-soluble organic acid (e.g., citric acid), inorganic salt (e.g. alkaline carbonate, an alkaline bicarbonate) and disintegrant (e.g., super disintegrant) make up granules. In some embodiments, the inorganic salt and/or the water-soluble organic acid may be in anhydrous form. In some embodiments, the disintegrant (e.g., super disintegrant) may be a sugar such as sorbitol. In some embodiments, the water-sensitive gas-generating formulation comprises a disintegrant (e.g., super disintegrant) and a viscosity enhancer.

In some embodiments, a gas-generating formulation may be an extended-release gas-generating formulation or effervescent. In some embodiments, the gas-generating formulation may include a viscosity enhancer. As used herein, a viscosity enhancer increase the viscosity of the composition comprising the first and second reactant. Suitable viscosity-increasing agents for use in accordance with this embodiment of the invention include, but are not limited to, cellulose derivatives (including, but not limited to, hydroxyethyl cellulose, carboxymethyl cellulose or its salts, hypromellose, and the like), polyvinylpyrrolidones (PVP) (preferably having a molecular weight of about 10,000 to about 350,000, as well as mixtures containing one or more grades or molecular weight of PVP), carrageenan, guar gum, alginates, carbomers, polyethylene glycols, polyvinyl alcohol, xanthan gum, and the like. In certain preferred embodiments, xanthan gum may be used as a viscosity-increasing agent.

For example, the viscosity enhancer may by extra granular, while a disintegrant (e.g., super disintegrant) may be intragranular. In some embodiments, the amount of the first and second reactants may be less than 80% of the single-unit, water-sensitive gas-generating formulation. In some embodiments, the water-sensitive gas-generating formulation may be a solid positioned adjacent (e.g., in contact with) to the water-permeable portion of the wall of the gas-generating reaction chamber. For example, the solid may be positioned adjacent (e.g., in contact with) to the at least two opposing surfaces of the wall which are water permeable. In some embodiments, the solid water-sensitive gas-generating formulation may be a multi-layer tablet, bilayer tablet, minitablet or micro tablet comprising two reactants. In some embodiments, the bilayer tablet comprises a first layer comprises a viscosity enhancer and a second layer having not a viscosity enhancer. In some embodiments, the solid water-sensitive gas-generating formulation may be configured to produce a consistent amount of gas over an extended period of time. In some embodiments, the extended period of time may be more than 30 minutes. In some embodiments, the solid water-sensitive gas-generating formulation comprises a viscosity enhancer.

In some embodiments, the water-sensitive gas-generating formulation may be in an amount sufficient to generate gas to provide a pressure of the inflatable patch as measured at 37° C. of about 2 to 5 psi. For example, the water-sensitive gas-generating formulation may be in an amount sufficient to generate gas to provide a consistent pressure of the inflatable patch as measured at 37° C. for about 30 minutes upon exposure of the gas-generating reaction chamber to water, intestinal fluid, or a model thereof.

In any of the embodiments described herein, the auto-inflatable patch assembly includes gas-generating formulation, wherein the molar ratio of acid; bicarbonate may be about 1:2 to 1:4. In any of the embodiments, the auto-inflatable patch assembly includes a gas-generating formulation having a molar ratio of acid: bicarbonate may be about 1:2, 1:3 or 1:4. In any of the embodiments, the auto-inflatable patch assembly includes the gas-generating formulation, wherein the molar ratio of citric acid:sodium bicarbonate may be about 1:2 to 1:4. In any of the embodiments, the auto-inflatable patch assembly includes a gas-generating formulation wherein the molar ratio of citric acid:sodium bicarbonate may be 1:2. In any of the embodiments, the auto-inflatable patch assembly includes a gas-generating formulation wherein the molar ratio of citric acid:sodium bicarbonate may be 1:3. In any of the embodiments, the auto-inflatable patch assembly includes a gas-generating formulation wherein the molar ratio of citric acid:sodium bicarbonate may be 1:4.

In any of the embodiments described herein, the auto-inflatable patch assembly includes a gas-generating formulation having a first and second reactant. In some embodiments, the amount of first and second reactant may be less than 80% by weight of the excipients in the gas-generating formulation. In some embodiments, the amount of first and second reactant may be less than 70% by weight of the excipients in the gas-generating formulation. In some embodiments, the amount of first and second reactant may be less than 65% by weight of the excipients in the gas-generating formulation. In some embodiments, the amount of first and second reactant may be about more than about 35% by weight of the excipients in the gas-generating formulation. In some embodiments, the amount of first and second reactant may be about more than about 50% by weight of the excipients in the gas-generating formulation. In some embodiments, the amount of first and second reactant may be about 35 to 70% by weight of the excipients in the gas-generating formulation.

In any of the embodiments described herein, the auto-inflatable patch assembly includes a gas-generating formulation which includes a first reactant in an amount of less than 40% by weight of the excipients in the gas-generating formulation. In any of the embodiments of the auto-inflatable patch assembly described herein, the gas-generating formulation can include a second reactant in an amount of less than 40% by weight of the excipients in the gas-generating formulation. In any of the embodiments of the auto-inflatable patch assembly described herein, the gas-generating formulation includes a ratio of filler to total reactants (e.g. first and second reactant) of about 20 to 35% or about 25% total reactants making up the gas-generating formulation.

In any of the embodiments described herein, the auto-inflatable patch assembly includes a gas-generating formulation which includes additional excipients. The choice of excipients should be carefully selected such they do not interfere but rather promote a gradual gas release upon exposure to water (e.g., gas or liquid form), are stable despite a degree of humidity in a typical environment and such that they promote an extended release of gas over time. The gradual release of gas contributes to the constant pressure over a period of time. Relevant excipients used in the preparation of the gas-generating formulation may include the following: a polymer, sugar, filler, lubricant, a super disintegrant, a polymer and binder. Typical fillers may include for example lactose, microcrystalline cellulose, starch, and mannitol. Typical disintegrants (e.g., super disintegrant) may include croscarmellose sodium, primogel, L-HPC, polyplasdone XL-10, crospovidone, plasdone XL, SSG primogel, LHPC, canmellose sodium, calcium carboxymethylcellulose, microcrystalline cellulose, alginic acid, alginates (e.g., sodium alginate, potassium alginate or calcium alginate), sodium starch glycolate, starch, and any mixtures thereof.

Additional choice and structure (e.g., layers) of excipients may be configured to generate an acidic local environment during the disintegration process for example with additional outer layer of acid. In some embodiments an acid can be used to increase acidity of the local environment during effervescence. In some examples, an outer coating or layer can be included to promote shelf stability due to the nature of the hygroscopic chamber wall housing the gas-generating formulation. In some examples, an outer coating or layer can be included to promote shelf stability due to the nature of the hygroscopic chamber wall housing the gas-generating formulation. In any of the embodiments of the auto-inflatable patch assembly described herein, the gas-generating formulation can include any one or more of the following: a sugar, bicarbonate, citric acid and PVP.

In any of the embodiments described herein, the auto-inflatable patch assembly includes a gas-generating formulation can be in any of a number of different forms of a typical single unit including but not limited to: multi-layer tablet, bilayer tablet, microparticles, thin film, minitablet, microtablet, granulate. In any of the embodiments described herein, the auto-inflatable patch assembly includes a gas-generating formulation can be in any of a number of different forms of a typical multi-unit formulation which can be retained by a formulation-retentive element including but not limited to: granules, pellets, microparticles, powder, minitablets, microtablets, granulate. In any of the embodiments of the auto-inflatable patch assembly described herein, the gas-generating formulation may be preferably a number of single unit micro or mini tablets. Typically, a microtablet size may be about 1.0 to 1.7 mm in diameter. Typically, a minitablet size may be about 1.8 to 3.5 mm in diameter. When granules, pellets, microparticles or a powder may be used, a formulation-retentive element.

In any of the embodiments described herein, the auto-inflatable patch assembly includes a gas-generating formulation can be film or layer within the gas-generating chamber. When it is a film or layer, it is possible that a formulation-retentive element is not necessary and a film or layer on gas-generating chamber inner surface is sufficient to retain the formulation within the gas-generating chamber.

In some embodiments, the single unit may be coated. In some examples, the single unit may be an additional layer on the inner gas-generating reaction chamber wall. When granulation is employed, an intra-granular or extra-granular superdisintegrant is present. In some embodiments, the single unit may have some degree of porosity. In some embodiments, the gas-generating formulation has true density of about 500 to 2500 mg/ml. In some embodiments, the gas-generating formulation has true density of about 1500 to 1900 mg/ml. In some embodiments, the gas-generating formulation has true density of about 1600 to 1700 mg/ml.

The shape of the single unit may be selected from any typical shape including for example a core, a rod, an oval, a round, a square, a rectangle, or a triangle shape. In any of the embodiments of the auto-inflatable patch assembly described herein, the gas-generating formulation has a shape to maximize a surface area to volume ratio, e.g., ring shape.

In any of the embodiments of the auto-inflatable patch assembly described herein, the gas-generating formulation can include a number of single units (e.g., mini tablets). In any of the embodiments of the auto-inflatable patch assembly described herein, the gas-generating formulation can include a number of single units (e.g., mini tablets), each containing either a first or second reactant. In any of the embodiments of the auto-inflatable patch assembly described herein, the first and second reactant are housed within the internal volume of the chamber in a multiple or single members. In any of the embodiments of the auto-inflatable patch assembly described herein, the first and second reactant are housed within the internal volume of the chamber in a multiple members. In any of the embodiments of the auto-inflatable patch assembly described herein, the first and second reactant are housed within the internal volume of the chamber in a single members. In any of the embodiments of the auto-inflatable patch assembly described herein, the gas-generating formulation can include an array of single units can be employed, each containing either a first or second reactant, wherein the array of units, each containing either a first or second reactant and are contain within a common gas-generating reaction chamber. In any of the embodiments of the auto-inflatable patch assembly described herein, the gas-generating formulation can include a number of single unit tablets can be employed, each containing either a first or second reactant wherein an array of tablets, each containing either a first or second reactant, may be contain in separate gas-generating reaction chambers.

In any of the embodiments of the auto-inflatable patch assembly described herein, the gas-generating formulation can have a total mass of more than 10 mg, 20 mg, 30 mg, 40 mg, 50 mg, 60 mg, 70 mg. In any of the embodiments of the auto-inflatable patch assembly described herein, the gas-generating formulation can have a total mass of less than 250 mg, less than 200 mg, 150 mg, 100 mg, or 90 mg. In any of the embodiments of the auto-inflatable patch assembly described herein, the gas-generating formulation can have a total mass of between 1 and 250 mg, 1 and 200 mg, 1 and 150, 1 to 150 mg, 50 to 150 mg or 70 to 100 mg per device. The total mass may be configured in one or multiple individual formulations including for example a single at least partial layer on the gas-generating inner wall or multiple tablets.

In any of the embodiments of the auto-inflatable patch assembly described herein, the gas-generating formulation can be manufactured in a single pot or multi-pot process. The formulation may be prepared by dry or wet granulation (e.g., in ethanol). The formulation may be a homogeneous mixture and or include multiple layers to have extended release of gas or additional shelf stability.

In any of the embodiments of the auto-inflatable patch assembly described herein, the gas-generating formulation may be configured to effervesce, i.e., release carbon dioxide, and completely disintegrate or degrade over a period of about 60 minutes. In any of the embodiments of the auto-inflatable patch assembly described herein, the gas-generating formulation may be configured to effervesce and completely disintegrate or degrade over a period of about 90 minutes. In any of the embodiments of the auto-inflatable patch assembly described herein, the gas-generating formulation may be configured to effervesce and completely disintegrate or degrade over a period of about 120 minutes.

In any of the embodiments of the auto-inflatable patch assembly described herein, the gas-generating formulation may be configured to generate a sufficient amount of gas within an internal volume of the inflatable patch to expand an exterior wall of the inflatable patch for a period of time to apply a sufficient penetration force as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 10 minutes to apply more than 1 pound per square inch as measured at 37° C. to advance at least a portion of the multiple tissue penetrating needle into the wall of an adjacent soft tissue.

In any of the embodiments of the auto-inflatable patch assembly described herein, the gas-generating formulation may be configured to generate a sufficient amount of gas within an internal volume of the inflatable patch to expand an exterior wall of the inflatable patch for a period of time to apply a sufficient penetration force as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 30 minutes to apply more than 1 pound per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 30 minutes to apply more than 2 pound per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 30 minutes to apply more than 3 pound per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 30 minutes to apply about 3.5 pound per square inch as measured at 37° C. to advance at least a portion of the multiple tissue penetrating needle into the wall of an adjacent soft tissue.

In any of the embodiments of the auto-inflatable patch assembly described herein, the gas-generating formulation may be configured to generate a sufficient amount of gas within an internal volume of the inflatable patch to expand an exterior wall of the inflatable patch for at least 45 minutes to apply more than 2 pound per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 45 minutes to apply more than 3 pound per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 45 minutes to apply about 3.5 pound per square inch as measured at 37° C. to advance at least a portion of the multiple tissue penetrating needle into the wall of an adjacent soft tissue.

In any of the embodiments of the auto-inflatable patch assembly described herein, the gas-generating formulation may be configured to generate a sufficient amount of gas within an internal volume of the inflatable patch to expand an exterior wall of the inflatable patch for at least 60 minutes to apply more than 2 pound per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 60 minutes to apply more than 3 pound per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 60 minutes to apply about 3.5 pound per square inch as measured at 37° C. to advance at least a portion of the multiple tissue penetrating needle into the wall of an adjacent soft tissue.

In any of the embodiments of the auto-inflatable patch assembly described herein, the gas-generating formulation may be configured to generate a sufficient amount of gas within an internal volume of the inflatable patch to expand an exterior wall of the inflatable patch for at least 60 minutes to apply 1 to 5 pounds per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 60 minutes to apply more than 1 to 4 pound per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 60 minutes to apply about 2 to 4 pound per square inch as measured at 37° C. to advance at least a portion of the multiple tissue penetrating needle into the wall of an adjacent soft tissue. In any of the embodiments of the auto-inflatable patch assembly described herein, the gas-generating formulation may be configured to generate a sufficient amount of gas within an internal volume of the inflatable patch to expand an exterior wall of the inflatable patch for at least 60 minutes to apply about 3 to 4 pound per square inch as measured at 37° C. to advance at least a portion of the multiple tissue penetrating needle into the wall of an adjacent soft tissue.

Typically, effervescent granules are manufactured in low humidity conditions. Care should be taken that all of the equipment used do not contain traces of water or moisture as it can destroy the effervescent reactant mixture. In final stages, a water-soluble lubricant can be included prior to compression into tablets. Strict control of humidity in all of the manufacturing areas may be necessary [25° C. less than 30% relative Humidity (RH)]. Any packaging material used for storage of the effervescent tablets should protect the tablet from external shear as well as entrap little amount of air within it since moisture present in entrapped air can lead to physical and/or chemical degradation of the tablet (Altomare et al., 1997).

In any of the embodiments described herein, the auto-inflatable patch assembly includes a fluid-only passage. A fluid-only passage, as used herein, includes any suitable passage adapted to pass gas that was generated in the gas-generating reaction chamber to an inflatable patch, while preventing passage of the gas-generating formulation which requires maintenance adjacent to a water-permeable surface. In some embodiments, the fluid-only passage may be operatively associated with a formulation-retentive element configured to retain a solid dosage form with the reaction chamber.

In any of the embodiments of the auto-inflatable patch assembly described herein, the fluid-only passage may be disposed between a gas-generating reaction chamber and an inflatable patch to facilitate a flow path for gas between an internal volume of the gas-generating reaction chamber and the internal volume of one or more inflatable patches. In any of the embodiments of the auto-inflatable patch assembly described herein, one or more gas-generating reaction chambers are fluidly connected to the inflatable patch via one or more fluid-only passages configured to connect between one or more gas-generating reaction chambers and the inflatable patch.

In some embodiments, one or more fluid-only passages are positioned along an outer circumference, perimeter, outer or side surface of the inflatable patch. For example, there may be two or more or three or more fluid-only passages positioned along an outer circumference, perimeter, outer or side surface of the inflatable patch. For example, there may be two to five fluid-only passages positioned along an outer circumference, perimeter, outer or side surface of the inflatable patch.

In any of the embodiments of the auto-inflatable drug delivery devices described herein, the fluid-only passage may include a valve on the internal volume to facilitate a direction of gas flow from the gas-generating reaction chamber towards the inflatable patch.

In any embodiment described herein, the auto-inflatable patch assembly can include an outer shell such as a swallowable enteric outer shell that houses or compresses the assembly. In some embodiments, the outer shell initially surrounds the auto-inflatable patch assembly prior to release in the intestine.

In any of the embodiments described herein, the auto-inflatable patch assembly can include a swallowable enteric outer shell sized to be swallowed and pass through the intestinal tract while also housing the auto-inflatable patch assembly. Examples of relevant sizes include but are not limited to 00-sized capsule (e.g., 00 elongated) or a 000-sized capsule. The outer shell defines an internal volume for housing the auto-inflatable patch assembly (i.e., gas-generating reaction chamber(s) and inflatable patch(s)). The outer shell can include a coating or a capsule, a coating over a capsule, or a capsule over a coating. Degradation of the outer shell may be in whole or in part, and may occur in stages. For example, the outer shell may include seams, perforations, or similar approaches to accelerate degradation or encourage early and complete exposure of the drug delivery device to the intestinal fluid.

The auto-inflatable patch assembly may be housed in a swallowable enteric outer shell, which degrades in whole or in part at, above, or below a design threshold, such as degrading when a pH level may be greater than 5.5. Breach of the outer shell due to degradation of the outer shell initiates a gas-generating process within the gas-generating chamber which culminates when drug may be deposited in the intestine wall via penetrating needles whether the therapeutic agent in liquid form may be being pushed out of the inflatable or by disintegrating or being deposited in a wall of a lumen through the penetrating needle for example of the GI tract such as the small intestine. The swallowable enteric outer shell can have at least a portion (e.g., coating or portion of the capsule), fabricated from various biocompatible polymers known in the art including various enteric polymers, which protects the outer shell from degradation in the stomach and degrades in response to a pH in the intestinal tract. For example, selective degradation may be initiated when exposed to a pH above about 5.5 to 6.0, commensurate to a pH level commonly found in the intestinal tract.

As used herein, “drug”, “active agent”, “therapeutic agent”, “therapeutic agent” or “medicine” may be used interchangeably and includes small molecules, biologics (antibody, protein, and peptide), diagnostics, or active pharmaceutical ingredients used to treat or diagnose a patient. They can refer to any agent that, when administered, has a therapeutic effect and/or elicits a desired biological and/or pharmacological effect. Generally, an therapeutic agent may refer to more than one therapeutic agent. Examples of therapeutic agents that may be suitable for use include but are not limited to chemotherapeutic agent, interferon, an antibody, an antibiotic, growth hormone, parathyroid hormone, a glucose regulating agent, an insulin compound, an incretin hormone, a GLP-1 compound or exenatide, antiviral, protease inhibitor or an anti-seizure compound or other therapeutic agents which would otherwise chemically degrade or have limited permeability if released within the intestine lumen of the gastrointestinal tract. In various embodiments of any of the aspects presented, the therapeutic agent is a peptide sequence, protein, an enzyme, a polysaccharide, or a polynucleotide, amino acids, nucleotides, carbohydrates, sugars, lipids, nucleoproteins, glycoproteins, lipoproteins, steroids, etc. whether naturally occurring or artificially created (e.g., by synthetic or recombinant methods) that are commonly found in cells and tissues. Specific classes of biologics include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

In any of the embodiments of the auto-inflatable patch assembly described herein, the therapeutic agent may be a polypeptide for example for digestion such as for example pancreatic polypeptide and/or glycopeptide metformin. The bioactive may be for example DPP4 inhibitors such as for example sitagliptin, vildagliptin, saxagliptin, linagliptin, gemigliptin, anagliptin, teneligliptin, alogliptin, trelagliptin, omarigliptin, evogliptin, dutogliptin and/or berberine lopeol. The bioactive may be for example sodium/glucose co-transporter 2 (SGLT2) inhibitors such as for example dapagliflozin, empaglifozin, canaglifocin, tofoglifocin, serglifocin, remoglifocin, ertuglifocin and/or sotaglifocin. The bioactive may be for example GLP-1 analogues such as for example incretins (e.g., semaglutide, glucagon-like peptide-1 (GLP-1), a GLP-1 analogue, exenatide, albiglutide, taspoglutide or a gastric inhibitory polypeptide (GIP)), efpeglenatid, exenatide, liraglutide, pramlintide, GnRH and analogs such as for example abarelix, cetrorelix, degarelix, ganirelix, elagolix relugolix, KLH-2109 and/or ASP-1707. The bioactive may be a protein which binds select transforming growth factor β superfamily ligands such as Luspatercept. The bioactive may be a protein blocks abnormal signaling between cells in the pulmonary blood vessels such as Sotatercept. The bioactive may be an anti-interleukin-13 (IL-13) monoclonal antibody such as Cendakimab. The bioactive may be other monoclonal antiboides such as Evinacumab, Ocrelizumab, Emicizumab, Dupilumab, Sarilumab, Isatuximab, Sutimlimab, Lanadelumab. The bioactive may be a selective inhibitor of TLR 7/8 such as afimetoran. For example, the therapeutic agent may be a Glucagon like peptide 1 receptor agonists or glucagon receptor agonist. Examples of therapeutic agents may include but are not limited by mazdutide, tirzepatide, cagrilintide semaglutide, pemvidutide, retatrutide, finopegdutide, bimagrumab or combinations thereof. The therapeutic agents may treat growth hormone deficiency such as somatrogon and lonapegsomatropin, Exenatide, liraglutide, lixisenatide, albiglutide and dulaglutide.

In any of the embodiments of the auto-inflatable patch assembly described herein, the therapeutic agent may be more than two different therapeutic agents which would benefit from similarly timed delivery via the intestinal wall.

In any of the embodiments of the auto-inflatable patch assembly described herein, the therapeutic agent may be a biological molecule (protein, peptide, etc.) or biological substance (e.g., cells) which would otherwise be chemically degraded by GI fluids in the GI tract (e.g. small intestine fluids) if not delivered into luminal wall of the GI tract. Such biological molecules may include various biologics which include one or more of proteins, antibodies, polypeptides and other molecules which are produced by cells or other biological process.

In any of the embodiments of the auto-inflatable patch assembly described herein, the therapeutic agent may be in an amount to produce a desired therapeutic effect in less than an amount to produce a corresponding effect if the agent was orally delivered without enclosure in the ingestible formulation. In some embodiments of any of the aspects presented, the therapeutic agent may be a combination of therapeutic agents. In some embodiments of any of the aspects presented, the therapeutic agent would chemically or enzymatically degrade, have limited permeability, or impose a deleterious effect on the subject if released within the lumen of the gastrointestinal tract and washed away. In some embodiments of any of the aspects presented, the therapeutic agent comprises a polypeptide that is chemically degraded or have limited permeability in the GI tract and the agent is delivered into the wall of the small intestine with minimal or no loss in binding affinity or specificity to a target binding site.

In any of the embodiments of the auto-inflatable patch assembly described herein, the therapeutic agent forms a portion of a solid penetration member. In any of the embodiments of the auto-inflatable patch assembly described herein, the therapeutic agent is a fluid or other non-solid form which is contained in a reservoir within the inflatable patch and which is advanced into the intestinal wall through one or more hollow needles or other injector by the gas applying a force on a reservoir of liquid dosage or other non-solid form. As used herein, fluid exhibits fluidic properties and may be in the form of a gas, liquid, colloidal suspension, gel slurry nano powder or powder.

As used herein, water impermeable refer to a structure being substantially impermeable to the to water of fluid, such that essentially no water or drug may be absorbed via the wall structure over a period of time.

It is to be understood that the term “gas impermeable” is a relative term. The compartments or chambers and specifically wall thereof must provide enough of a barrier to prevent substantial escape of gas to maintain the desired pressure inside the compartments or chambers. That being said, because there is continuous gas generation and varying degree of gas pressure applied from the internal volume to the wall which is continuously absorbing water, there is some degree of tolerance to the escape of gas.

It is to be understood that the term “stable” is a relative term. It is desired that a stable device and formulation be stable during the desired shelf life of the packaged device assembly. The details of the properties required for this goal will depend on the length of the desired shelf life, the amount of vapor-donating liquid placed in the package before scaling the package from the environment, and the conditions under which the product is stored.

The terms “substantially” and “about” are used herein to describe and account for insignificant variations. For example, when used in association with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

As used in the specification and the appended claims, the singular forms “a,” “an” and “the” include plural equivalents unless the context clearly indicates otherwise. Thus, for example, reference to “a disintegrant” can include mixtures of two or more disintegrants.

Additionally, amounts, ratios, and other numerical values may sometimes be presented herein in a range format. As used herein, a range of numbers includes any number within the range or any sub-range if the minimum and maximum numbers in the sub-range fall within the scope. Ranges can be expressed herein as from one particular value, and/or to another particular value. When such a range is expressed, another aspect includes from the one particular value and/or to the other particular value. It will be further understood that the endpoints of each of the ranges are significant both in relation to the other endpoint, and independently of the other endpoint. For example, if “1 to 6” is disclosed, and 2 to 4 is disclosed, then 4 to 6 are also disclosed. It is also understood that there are a number of values disclosed herein, and that each value is also herein disclosed as “about” that particular value in addition to the value itself. For example, if the value “10” is disclosed, then “about 10” is also disclosed. It is also understood that whenever a range is recited within this application, every whole number integer within the range is also contemplated as an embodiment of the invention and vice versa. For example, if a list of units are provided, multiple ranges between the units provided are also contemplated. CLAIMS

1. An auto-inflatable patch assembly for drug delivery within a lumen, comprising:

i. a pliable and inflatable patch having water and gas-impermeable walls, which define an internal chamber and an array of penetrating needles disposed on a surface thereof;
ii. one or more auxiliary gas-generating reaction chambers distinguished from and fluidly connected to the inflatable patch said reaction chamber defined by pliable reaction chamber walls that house a water-sensitive gas-generating formulations; said reaction chamber walls configured to constrain gas and having at least a portion being a water-permeable outer surface,
wherein the patch assembly is configured such that each gas-generating formulation or a set thereof, is retained within its respective auxiliary gas-generating reaction chamber during inflation.

2. The auto-inflatable patch assembly of claim 1, wherein the patch assembly is compressed and further housed within a swallowable enteric outer shell.

3. The auto-inflatable patch assembly of any one of claims 1 to 2, wherein the water-sensitive gas-generating formulation is an extended-release gas-generating formulation.

4. The auto-inflatable patch assembly of any one of claims 1 to 3, wherein the water-sensitive gas-generating formulation is configured to generate gas to provide a pressure of the inflatable patch as measured at 37° C. of more than 3 psi.

5. The auto-inflatable patch assembly of any one of claims 1 to 4, wherein the water-sensitive gas-generating formulation is configured to generate gas to provide a substantially consistent pressure of the inflatable patch as measured at 37° C. for about 30 minutes.

6. The auto-inflatable patch assembly of claim 3, wherein the extended-release gas-generating formulation includes a viscosity enhancer.

7. The auto-inflatable patch assembly of any one of claims 1 to 6, wherein one or more auxiliary gas-generating reaction chambers are fluidly connected to the internal chamber but separated from the inflation patch by a formulation-retentive element.

8. The auto-inflatable patch assembly of any one of claims 1 to 7, wherein the patch assembly is configured so that a combination of the internal chamber of the patch and respective interiors of the one or more auxiliary gas reaction chambers are configured to constrain gas within.

9. The auto-inflatable patch assembly of any one of claims 1 to 8, wherein an amount of pressure produced within the gas-generating reaction chamber does not cause substantial gas leakage from the auto-inflatable patch for a first period.

10. The auto-inflatable patch assembly of any one of claims 1 to 9, wherein the first and second reactants are housed within a single compartment.

11. The auto-inflatable patch assembly of any one of claims 1 to 10, wherein the gas-generating reaction chamber is positioned along an outer circumference, perimeter, or side surface of the inflatable patch.

12. The auto-inflatable patch assembly of any one of claims 1 to 11, wherein three or more gas-generating reaction chambers are positioned along an outer circumference, perimeter, or side surface of the inflatable patch.

13. The auto-inflatable patch assembly of any one of claims 1 to 12, wherein a formulation-retentive element is positioned along an inflatable patch outer circumference, perimeter, outer or side surface.

14. The auto-inflatable patch assembly of any one of claims 1 to 13, wherein two or more formulation-retentive elements are positioned along an outer circumference, perimeter, outer or side surface of the inflatable patch.

15. The auto-inflatable patch assembly of any one of claims 1 to 14, wherein three or more formulation-retentive elements are positioned along an outer circumference, perimeter, outer or side surface of the inflatable patch.

16. The auto-inflatable patch assembly of any one of claims 1 to 15, wherein two to five formulation-retentive elements are positioned along an outer circumference, perimeter, outer or side surface of the inflatable patch.

17. The auto-inflatable patch assembly of any one of claims 1 to 10, wherein the gas-generating reaction chamber is surrounded by the inflatable patch and the formulation-retentive element is positioned along an outer circumference, perimeter, outer or side surface of one or more gas-generating reaction chambers.

18. The auto-inflatable patch assembly of any one of claims 1 to 17, wherein the gas-generating reaction chamber is configured to produce a consistent amount of gas over an extended period.

19. The auto-inflatable patch assembly of claim 18, wherein the extended period is more than 30 minutes.

20. The auto-inflatable patch assembly of any one of claims 1 to 19, wherein at least 50% of the outer surface of the reaction chamber wall is water-permeable.

21. The auto-inflatable patch assembly of any one of claims 1 to 20, wherein at least a portion of the reaction chamber walls are water absorbing.

22. The auto-inflatable patch assembly of any one of claims 1 to 21, wherein the reaction chamber walls are directionally permeable.

23. The auto-inflatable patch assembly of any one of claims 1 to 22, wherein the penetrating needles have a length of more than 6.0 mm.

24. The auto-inflatable patch assembly of any one of claims 1 to 23, wherein penetrating needles have a length of between 1.4 and 2.8 mm.

25. The auto-inflatable patch assembly of any one of claims 23 to 24 wherein penetrating needles further comprise a therapeutic agent.

26. The auto-inflatable patch assembly of any one of claims 23 to 25, wherein the inflatable patch comprises a fluid therapeutic agent operably connected to a penetrating needle base.

27. The auto-inflatable patch assembly of any one of claims 1 to 26, wherein the array of penetrating needles are positioned on a first surface of the inflatable patch.

28. The auto-inflatable patch assembly of any one of claims 1 to 27, wherein the array of penetrating needles are positioned on a planar surface of the inflatable patch in its uninflated expanded state.

29. The auto-inflatable patch assembly of any one of claims 1 to 28, wherein the array of penetrating needles are positioned on the first surface of the inflatable patch, and the inflatable patch further comprises an opposing wall having a surface, wherein the surface has no penetrating needles.

30. The auto-inflatable patch assembly of any one of claims 1 to 29, wherein the inflatable patch has a maximum dimension of less than 9 cm.

31. The auto-inflatable patch assembly of claim 30, wherein the inflatable patch has a maximum dimension of less than 5.5 cm.

32. The auto-inflatable patch assembly of any one of claims 1 to 31, wherein the inflatable patch does not have a water permeable surface.

33. The auto-inflatable patch assembly of any one of claims 1 to 32 wherein the inflatable patch is shaped and sized for contacting a partial inner circumference of the intestine.

34. The auto-inflatable patch assembly of any one of claims 1 to 33, wherein a compressed-state auto-inflatable patch assembly comprises an auxiliary gas-generating reaction chambers positioned on an outer surface of the compressed state auto-inflatable patch.

35. An auto-inflatable patch assembly comprising: a pliable and inflatable patch having water and gas-impermeable walls which define an internal chamber; one or more auxiliary gas-generating reaction chambers distinguished from and fluidly connected to the inflatable patch, said reaction chamber defined by pliable reaction chamber walls that house a first and second reactant in a single compartment, said first and second reactants configured to effervesce when exposed to water; said reaction chamber walls configured to constrain gas and having at least a portion being a water-permeable outer surface.

36. The auto-inflatable patch assembly of claim 35, wherein a single compartment is separated from the inflatable patch by a formulation-retentive element.

37. The auto-inflatable patch assembly of any one of claims 35 to 36, wherein the patch assembly further comprises an array of penetrating needles disposed on a surface of the inflatable patch.

38. The auto-inflatable patch assembly of any one of claims 35 to 37, wherein the water-sensitive gas-generating formulation is configured for extended-release.

39. The auto-inflatable patch assembly of any one of claims 35 to 38, wherein the water-sensitive gas-generating formulation comprises a viscosity enhancer.

40. The auto-inflatable patch assembly of any one of claims 35 to 39, wherein the water-sensitive gas-generating formulation comprises a viscosity enhancer which makes up an extra-granular portion.

41. The auto-inflatable patch assembly of claim 40, wherein a citric acid, alkaline carbonate, and a viscosity enhancer make up an extra-granular portion.

42. The auto-inflatable patch assembly of any one of claims 35 to 41, wherein a water-sensitive gas-generating formulation is a single unit.

43. The auto-inflatable patch assembly of any one of claims 35 to 42 wherein the water-sensitive gas-generating formulation is a solid dosage form.

44. The auto-inflatable patch assembly of any one of claims 35 to 43, wherein the amount of first and second reactant amount is less than 80% of the water-sensitive gas-generating formulation.

45. The auto-inflatable patch assembly of any one of claims 35 to 44, wherein the first and second reactant are in anhydrous form.

46. The auto-inflatable patch assembly of any one any of claims 35 to 45, wherein the disintegrant is a sugar.

47. The auto-inflatable patch assembly of claim 46, wherein the sugar is sorbitol.

48. The auto-inflatable patch assembly of any one any of claims 35 to 47, wherein the gas-generating reaction chamber comprises two or more water-permeable surfaces.

49. The auto-inflatable patch assembly of any one any of claims 35 to 48, wherein the gas-generating reaction chamber comprises two or more opposing water-permeable surfaces.

50. A method for providing an auto-inflatable patch assembly for delivering a therapeutic agent into an intestinal wall of a subject comprising:
a. orally administering to the subject a device comprising a swallowable enteric outer shell and an auto-inflatable patch assembly, said patch assembly comprising a pliable and inflatable patch having water and gas-impermeable walls, which define an internal chamber and an array of penetrating needles disposed on a surface thereof, auxiliary gas-generating reaction chambers defined by pliable reaction chamber walls that house a water-sensitive gas-generating formulation; said reaction chamber walls configured to constrain gas and having at least a portion being a water-permeable outer surface;
b. exposing a water-sensitive gas-generating formulation to water through a water-permeable outer surface of the gas-generating reaction chamber such that the formulation generates an extended release of gas that flows towards the inflatable patch while being retained within its respective auxiliary gas-generating reaction chamber.

51. A method for making a auto-inflatable patch assembly for delivering a therapeutic agent into an intestinal wall of a subject comprising: forming an extended-release water-sensitive gas-generating formulation; providing an upper and lower water and gas-impermeable layer, said layer having a portion being a water-permeable surface; positioning a water-sensitive gas-generating formulation adjacent to the water-permeable surface and further providing a formulation-retentive element between the formulation and the inflatable patch such that each gas-generating formulation is retained within its respective auxiliary gas-generating reaction chamber; sealing a seam connecting the upper and lower layers to form an auto-inflatable patch; folding or rolling the auto-inflatable patch assembly within a capsule wherein water-permeable surface is maintained on an outer surface of the compressed state auto-inflatable patch.
